(19) United States
(12) Patent Application Publication
(10) Pub. No.: US 2024/0398253 A1
(43) Pub. Date: Dec. 5, 2024

(54) Physics-based algorithm to universally standardize routinely obtained clinical T2-weighted images

(71) Applicant: YALE UNIVERSITY, New Haven, CT (US)

(72) Inventors: Gigi Galiana Stadtler, Guilford, CT (US); Hemant Tagare, North Haven, CT (US)

(21) Appl. No.: 18/734,158

(22) Filed: Jun. 5, 2024

Related U.S. Application Data
(60) Provisional application No. 63/506,230, filed on Jun. 5, 2023.

(51) Int. Cl. A61B 5/055; G01R 33/48; G01R 33/50; G01R 33/56

(52) U.S. Cl. CPC A61B 5/055; G01R 33/482; G01R 33/50; G01R 33/5602; G01R 33/5608

(57) ABSTRACT

An imaging method calculates T₂ maps using only data from a T₂w image scan. The imaging system is used in conjunction with an MRI system that includes a magnet housing, a superconducting magnet, shim coils, RF coils, receiver coils, a patient support, and measurement circuitry producing data used to reconstruct images displayed on a display. The measurement circuitry integrates either expanding-constrained alternating minimization for parameter mapping or expanding-constrained alternating minimization for parameter mapping with projected gradient descent.

### CROSS REFERENCE TO RELATED APPLICATION

The application claims the benefit of U.S. Provisional Patent Application Ser. No. 63/506,230, entitled “A physics-based algorithm to universally standardize routinely obtained clinical T2-weighted images,” filed Jun. 5, 2023, which is incorporated herein by reference.

### BACKGROUND OF THE INVENTION

### 1. Field of the Invention

The present invention relates to an imaging method and system calculating T₂ maps.

### 2. Description of the Related Art

Magnetic resonance (MR) images reflect important biology, but the information in these images is also affected by the details of how the images are acquired, such as MRI (Magnetic Resonance Imaging) hardware, protocol settings, etc. Variations in these parameters make it difficult to train effective and generalizable AI (Artificial Intelligence) for MR image analysis because an AI algorithm trained on data from one site may not perform well on data from a different site.

Images from routine clinical MRI scans could be a massive data source for machine learning, with nearly 40 million MRI studies conducted annually in the U.S. Smith-Bindman Rebecca, Kwan Marilyn L., Marlow Emily C., et al. Trends in Use of Medical Imaging in US Health Care Systems and in Ontario, Canada, 2000-2016. JAMA. 2019; 322(9):843-856. The vast majority of the clinical protocols produce qualitative images whose contrast and intensity are subject to the scanner hardware and scanning protocol. Cashmore Matt T., McCann Aaron J., Wastling Stephen J., McGrath Cormac, Thornton John, Hall, Matt G. Clinical quantitative MRI and the need for metrology. The British Journal of Radiology. 2021; 94(1120):20201215. This heterogeneity hinders the direct translation of routine clinical MR images into large-scale datasets, and image standardization that removes scanner- and/or protocol specific variance is required. Twari Pallavi, Verma Ruchika. The Pursuit of Generalizability to Enable Clinical Translation of Radiomics. Radiology: Artificial Intelligence. 2021; 3(1):e200227. Publisher: Radiological Society of North America; Onofrey John A., Casetti-Dinescu Dana I., Lauritzen Andreas D., et al. Generalizable Multi-Site Training and Testing Of Deep Neural Networks Using Image Normalization. In: 2019 IEEE 16th International Symposium on Biomedical Imaging (ISBI 2019):348-351; 2019; Venice, Italy. A universal approach to standardization is to reveal the underlying quantitative physical properties that are invariant across scans, e.g. relaxivity. Instead of time-consuming quantitative mapping, reconstructing quantitative maps from routine clinically acquired data obviates changes to scanning protocols and provides standardized images.

The Turbo Spin Echo (TSE) is a common sequence included in most clinical protocols. To improve smoothness in k-space, acquisitions at the same echo time usually sample adjacent lines of k-space that constitute a band, with each band corresponding to a certain echo time (TE). This sampling pattern is named as band-sampling. Elsaid Nahla M. H., Tagare Hemant D., Galiana Gigi. A Physics-Based Algorithm to Universally Standardize Routinely Obtained Clinical T2-Weighted Images. Academic Radiology. 2024; 31(2):582-595. The multi-echo data follows exponential T₂-decay and the direct image reconstruction of k-space collapsed over the TE-dimension generates T₂-weighted (T₂w) images. However, the standardization of clinical TSE data by generating the T₂ map is not trivial. State-of-the-art methods for undersampled reconstruction of T₂ maps rely on trajectories that sample the k-space center at every echo, ensuring a relatively uniform intensity across echoes. Wang Xiaoqing, Tan Zhengguo, Scholand Nick, Roeloffs Volkert, Uecker Martin. Physics-based reconstruction methods for magnetic resonance imaging. Philosophical Transactions of the Royal Society A: Mathematical, Physical and Engineering Sciences. 2021; 379(2200):20200196. Publisher: Royal Society; Huang Chuan, Graff Christian G., Clarkson Eric W., Bilgin Ali, Altbach Maria I. T₂ mapping from highly undersampled data by reconstruction of principal component coefficient maps using compressed sensing. Magnetic Resonance in Medicine. 2012; 67(5):1355-1366; Block K. T., Uecker M., Frahm J. Model-based iterative reconstruction for radial fast spin-echo MRI IEEE Transactions on Medical Imaging. 2009; 28(11):1759-1769. Direct translation of these algorithms to clinical TSE data is hindered by the significantly different intensity across echoes due to the band-sampling pattern, which overshadows the signal evolution model (as seen in FIG. 1 of Elsaid Nahla M. H., Tagare Hemant D., Galiana Gigi. A Physics-Based Algorithm to Universally Standardize Routinely Obtained Clinical T2-Weighted Images. Academic Radiology. 2024; 31(2):582-595).

Magnetic Resonance Imaging (MRI) machine learning (ML)/artificial intelligence (AI) requires careful standardization of large image datasets. P. Lambin, R. T. H. Leijenaar, T. M. Deist, J. Peerlings, E. E. C. de Jong, J. van Timmeren, S. Sanduleanu, R. Larue, A. J. G. Even, A. Jochems, Y. van Wijk, H. Woodruff, J. van Soest, T. Lustberg, E. Roelofs, W. van Elmpt, A. Dekker, F. M. Mottaghy, J. E. Wildberger, and S. Walsh, Radiomics: the bridge between medical imaging and personalized medicine, Nat Rev Clin Oncol, vol. 14, no. 12, pp. 749-762, December, 2017; T. Jo, K. Nho, and A. J. Saykin, Deep Learning in Alzheimer's Disease: Diagnostic Classification and Prognostic Prediction Using Neuroimaging Data, Frontiers in Aging Neuroscience, vol. 11, 2019—August—481 20, 2019; W. Yassin, H. Nakatani, Y. Zhu, M. Kojima, K. Owada, H. Kuwabara, W. Gonoi, Y. Aoki, H. Takao, T. Natsubori, N. Iwashiro, K. Kasai, Y. Kano, O. Abe, H. Yamasue, and S. Koike, Machine-learning classification using neuroimaging data in schizophrenia, autism, ultra-high risk and first-episode psychosis, Translational Psychiatry, vol. 10, no. 1, pp. 278, 2020/08/17, 2020. One source of standardized datasets is MRI studies, in which the acquisition variables are carefully controlled across machines and sites. D. C. Van Essen, S. M. Smith, D. M. Barch, T. E. Behrens, E. Yacoub, and K. Ugurbil, The WU-Minn Human Connectome Project: an overview, Neuroimage, vol. 80, pp. 62-79, Oct. 15, 2013.488; T. Sarwar, Y. Tian, B. T. T. Yeo, K. Ramamohanarao, and A. Zalesky, Structure-function coupling in the 489 human connectome: A machine learning approach, NeuroImage, vol. 226, pp. 117609, 2021 Feb. 1, 2021. Unfortunately, such datasets are expensive to acquire, not only for the cost of custom-ordered scanning but also for the painstaking organizational and administrative efforts needed to ensure a uniform acquisition protocol.

An alternative source of images for ML/AI is clinical exams (˜30 million annually in the US alone). The challenge with using clinical MRI for ML/AI is that most clinical scans are not quantitative, so variations in image intensity depend not only on the biological parameters of interest (e.g., T₂ or proton density), but also on hardware and acquisition variables. P. Twari, and R. Verma, The Pursuit of Generalizability to Enable Clinical Translation of Radiomics, Radiol Artif Intell, vol. 3, no. 1, pp. e200227, January 2021; P. Chirra, P. Leo, M. Yim, B. N. Bloch, A. R. Rastinehad, A. Purysko, M. Rosen, A. Madabhushi, and S. E. Viswanath, Multisite evaluation of radiomic feature reproducibility and discriminability for identifying peripheral zone prostate tumors on MRI, J Med Imaging (Bellingham), vol. 6, no. 2, pp. 024502, April, 2019; H. Um, F. Tixier, D. Bermudez, J. O. Deasy, R. J. Young, and H. Veeraraghavan, Impact of image preprocessing on the scanner dependence of multi-parametric MRI radiomic features and covariate shift in multi-institutional glioblastoma datasets, Phys Med Biol, vol. 64, no. 16, pp. 165011, Aug. 21, 2019. These variations have been shown to significantly impact AI algorithm performance, even for tasks, such as organ segmentation, that would be expected to rely on intra-image contrast. J. A. Onofrey, D. I. Casetti-Dinescu, A. D. Lauritzen, S. Sarkar, R. Venkataraman, R. E. Fan, G. A. Sonn, P. C. Sprenkle, L. H. Staib, and X. Papademetris, Generalizable Multi-Site Training And Testing Of Deep Neural Networks Using Image Normalization, Proc IEEE Int Symp Biomed Imaging, vol. 2019, pp. 348-351, April, 2019.

Several methods have been proposed to harmonize images, i.e., remove site-dependent intensity variations that do not reflect biologically important contrast. However, these methods decide apriori which image features should be uniform across the dataset. R. T. Shinohara, E. M. Sweeney, J. Goldsmith, N. Shiee, F. J. Mateen, P. A. Calabresi, S. Jarso, D. L. Pham, D. S. Reich, and C. M. Crainiceanu, Statistical normalization techniques for magnetic resonance imaging, Neuroimage Clin, vol. 6, pp. 9-19, 2014; W. E. Johnson, C. Li, and A. Rabinovic, Adjusting batch effects in microarray expression data using empirical Bayes methods, Biostatistics, vol. 8, no. 1, pp. 118-127, 2006; J.-P. Fortin, D. Parker, B. Tune, T. Watanabe, M. A. Elliott, K. Ruparel, D. R. Roalf, T. D. Satterthwaite, R. C. Gur, R. E. Gur, R. T. Schultz, R. Verma, and R. T. Shinohara, Harmonization of multi-site diffusion tensor imaging data, NeuroImage, vol. 161, pp. 149-170, 2017 Nov. 1, 2017. The principles of nuclear magnetism: Oxford University. As a result, there is no guarantee that a given harmonization strategy will be appropriate for a new dataset or learning task, and it is also possible to wash away true biological variability.

Quantitative MRI (qMRI) methods solve directly for the biologically relevant physical parameters that are captured in a typical MR image. These method have been shown to remove acquisition dependence while retaining all MR-sensitive biological variability, so they are an ideal alternative to image-based harmonization. However, quantitative MRI requires specialized sequences, which has limited its adoption into routine clinical practice. On a national scale, a very small fraction of MRI exams includes a T₂ mapping scan that is commonly used to calculate the T₂ times of a certain tissue and display them voxel-vice on a parametric map, while nearly all include some kind of T₂w (T₂ weighted) image scan that relies upon the transverse relaxation of the net magnetization vector.

Conventional quantitative T₂ mapping uses a series of images (see for example those shown in FIG. 1(a)) acquired with different timings after excitation (echo times, or TE) to fit a physics model across time for each pixel:

$${I}_{TE} ( x ) = {I}_{0} ( x ) {e}^{- \frac{TE}{{T}_{2} ( x )}} ,$$

where I_{TE}(x) is the image brightness at a given TE, I₀(x) is the image brightness at TE=0, and T₂(x) is a constant that reflects the tissue microstructure in that voxel. A. Abragam, The principles of nuclear magnetism: Oxford university press, 1961. As discussed below, this model can act as a physics-based mathematical constraint in the reconstruction algorithm.

The simplest form of T₂ mapping acquires an echo train sampling the full k-space of magnetization at each echo time. Because this approach requires long scan times, many groups have shown the feasibility of generating T₂ maps and images from accelerated experiments, where only a fraction of k-space is acquired at echo, typically in distributed interleaved patterns that favor parallel imaging reconstruction of the individual echo images while providing complementary spatial information across echoes (FIG. 1c). M. A. Cloos, F. Knoll, T. Zhao, K. T. Block, M. Bruno, G. C. Wiggins, and D. K. Sodickson, Multiparametric imaging with heterogeneous radiofrequency fields, Nature Communications, vol. 7, no. 1, pp. 12445, 2016/08/16, 2016; K. T. Block, M. Uecker, and J. Frahm, Model-based iterative reconstruction for radial fast spin-echo MRI, IEEE transactions on medical imaging, vol. 28, no. 11, pp. 1759-1769, 2009 November, 2009; T. J. Sumpf, A. Petrovic, M. Uecker, F. Knoll, and J. Frahm, Fast T₂ Mapping With Improved Accuracy Using Undersampled Spin-Echo MRI and Model-Based Reconstructions With a Generating Function, IEEE Transactions on Medical Imaging, vol. 33, no. 12, pp. 2213-2222, 2014; C. Huang, C. G. Graff, E. W. Clarkson, A. Bilgin, and M. I. Altbach, T₂ mapping from highly undersampled data by reconstruction of principal component coefficient maps using compressed sensing, Magn Reson Med, vol. 67, no. 5, pp. 1355-66, May, 2012; G. Nataraj, J. F. Nielsen, C. Scott, and J. A. Fessler, Dictionary-Free MRI PERK: Parameter Estimation via Regression with Kernels, IEEE Trans Med Imaging, vol. 37, no. 9, pp. 2103-2114, September, 2018; F. H. Petzschner, I. P. Ponce, M. Blaimer, P. M. Jakob, and F. A. Breuer, Fast MR parameter mapping using k-t principal component analysis, Magn Reson Med, vol. 66, no. 3, pp. 706-16, September, 2011; J. I. Tamir, M. Uecker, W. Chen, P. Lai, M. T. Alley, S. S. Vasanawala, and M. Lustig, T₂ shuffling: Sharp, multicontrast, volumetric fast spin-echo imaging, Magnetic Resonance in Medicine, vol. 77, no. 1, pp. 180-528 195, 2017. Under some circumstances, these accelerated sequences can be made as short as traditional T₂w scans, which they could practically replace, but this has not happened on a national scale.

Similar to accelerated T₂ mapping, clinical T₂w imaging (T₂w-TSE), which is included in most MR exams, also acquires an echo train with different and complementary lines of k-space at each echo time (FIG. 1(d( ). These are typically combined to fill a single k-space, whose reconstruction is an image where brightness qualitatively reflects T₂, aka a T₂w image. J. Hennig, A. Nauerth, and H. Friedburg, RARE imaging: A fast imaging method for clinical MR, Magnetic Resonance in Medicine, vol. 3, no. 6, pp. 823-833, 1986. However, the data shown in FIG. 1d can also be thought of as an undersampled T₂ mapping experiment with a different k-space sampling pattern, which we call “band-sampled k-space” to distinguish it from the interleaved sampling patterns used in previous work. While previous methods have hypothesized a connection between T₂w images and T₂ mapping, they fail to reconstruct actual T₂w image data as an undersampled T₂ mapping experiment.

While the k-space sampling patterns in FIG. 1(c) and FIG. 1(d) both represent complementary samplings of k-space across echoes, previous reconstruction methods cannot handle the narrow span of k-space sampled at each echo in FIG. 1(d). This is demonstrated in FIG. 2, where the first row shows the ground truth from fully sampled spin echo data at different echo times. Subsequent rows show reconstructions from data where only non-overlapping strips of k-space are available at each echo, as is the case in a T₂w-TSE image acquisition.

FIG. 2(d) shows that, without advanced reconstruction methods, i.e., only enforcing data consistency between the data and image at each echo, the brightness of each image is dominated by the uneven sampling energy across different parts of k-space. More sophisticated methods improve reconstruction by imposing a model that relates images at different echo times, which effectively shares information across images. Two modern examples of these are shown in FIGS. 2(c) and 2(d), using a basis-constrained and model-based reconstruction, respectively. C. Huang, C. G. Graff, E. W. Clarkson, A. Bilgin, and M. I. Altbach, T₂ mapping from highly undersampled data by reconstruction of principal component coefficient maps using compressed sensing, Magn Reson Med, vol. 67, no. 5, pp. 1355-66, May, 2012; X. Wang, Z. Tan, N. Scholand, V. Roeloffs, and M. Uecker, Physics-based reconstruction methods for magnetic resonance imaging, Philosophical Transactions of the Royal Society A: Mathematical, Physical and Engineering Sciences, vol. 379, no. 2200, pp. 20200196, 2021. In both cases, the physics constraint clearly promotes data sharing across images, improving individual reconstructions. However, the T₂ map is entirely inaccurate.

### SUMMARY OF THE INVENTION

It is, therefore, an object of the present invention to provide an imaging method that calculates T₂ maps using only data from a T₂w image scan.

In some embodiments calculating uses an exact but linear expression to capture an exponential decay model.

In some embodiments calculating is biconvex so that it can be alternately minimized.

In some embodiments calculating applies a stepwise initialization process that avoids spurious local minima due to the significant gaps in k-space.

In some embodiments calculating includes a signal evolution model constraint that is enforced directly by projected gradient descent.

In some embodiments calculating includes virtual conjugate coils.

It is another object to provide an MRI system that includes a magnet housing, a superconducting magnet, shim coils, RF coils, receiver coils, a patient support, and measurement circuitry producing data used to reconstruct images displayed on a display. The measurement circuitry integrates either expanding-constrained alternating minimization for parameter mapping or expanding-constrained alternating minimization for parameter mapping with projected gradient descent.

In some embodiments expanding-constrained alternating minimization for parameter mapping or expanding-constrained alternating minimization for parameter mapping with projected gradient descent calculates T₂ maps using only data from the T₂w image scan.

In some embodiments expanding-constrained alternating minimization for parameter mapping or expanding-constrained alternating minimization for parameter mapping with projected gradient descent uses an exact but linear expression to capture an exponential decay model.

In some embodiments expanding-constrained alternating minimization for parameter mapping or expanding-constrained alternating minimization for parameter mapping with projected gradient descent is biconvex so that it can be alternately minimized.

In some embodiments expanding-constrained alternating minimization for parameter mapping or expanding-constrained alternating minimization for parameter mapping with projected gradient descent applies a stepwise initialization process that avoids spurious local minima due to the significant gaps in k-space.

In some embodiments expanding-constrained alternating minimization for parameter mapping with projected gradient descent includes a signal evolution model constraint that is enforced directly by projected gradient descent.

In some embodiments expanding-constrained alternating minimization for parameter mapping with projected gradient descent includes virtual conjugate coils.

Other objects and advantages of the present invention will become apparent from the following detailed description when viewed in conjunction with the accompanying drawings, which set forth certain embodiments of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1, which is composed of FIGS. 1(a) to 1(e), shows a framework of the e-CAMP algorithm with four mₚs, where the color of the k-space lines represents acquisition at a different ΔTE, where FIG. 1(a) shows ground truth echo images, FIG. 1(b) shows fully sampled k-space, FIG. 1(c) shows k-space of accelerated echo images, and FIG. 1(d) shows k-space of standard TSE image. FIG. 1(e) shows the e-CAMP iterations: in cycle 1, e-CAMP applies the CAMP algorithm to center bands until it converges. Then e-CAMP grows the number of mps to include 3 bands (cycle 2) and finally, it includes all the bands (cycle 3).

FIG. 2, which is composed of FIGS. 2(a) to 2(e), shows T₂ maps and mₚs of: FIG. 1(a) shows fully sampled spin echo images (ground truth), FIG. 1(b) shows no model, FIG. 1(c) shows subspace constrained, FIG. 1(d) shows model-based, and FIG. 1(e) shows e-CAMP reconstructions.

FIG. 3 shows simulation results showing the ground truth T₂ maps and T₂w images versus the maps and T₂w images reconstructed using e-CAMP, with the difference image and the masked absolute difference (showing errors inside the brain). In addition, a regression analysis is plotted between the e-CAMP T₂ map versus that of the ground truth.

FIG. 4, which is composed of FIGS. 4(a) to 4(d), shows retrospective and prospective human brain experiments with: FIG. 4(a) shows four-echo results (25 ms echo spacing), and FIG. 4(b) shows four-echo results (40 ms) of a different subject. FIG. 4(c) shows eight-echo results (20 ms echo spacing) from the same MESE acquisition of FIG. 4(b) showing the fully sampled and e-CAMP T₂ maps, T₂w images, absolute difference, and normalized versions of the absolute difference.

Similarly, FIG. 4(d) shows prospective human brain experiments from nine-echo experiments with 20 ms echo spacing, showing the fully sampled and e-CAMP T₂ maps, T₂w images, absolute difference, and normalized versions of the absolute difference.

FIG. 5 shows prospective human brain experiments with nine-echo results with 20 ms echo spacing showing ground truth SE T₂ map versus fully sampled TSE T₂ map and e-CAMP T₂ map with their corresponding error maps. The lower row shows the EMC (Echo Modulation Curve) corrected versions of the fully sampled T₂ map and e-CAMP T₂ map with their corresponding error maps.

FIG. 6 shows prospective phantom experiments ground truth SE T₂ map versus fully sampled MESE T₂ map and e-CAMP T₂ map.

FIG. 7, which is composed of FIGS. 7(a) to 7(d), shows using the segmentation map shown in FIG. 7(a) comparison between the true T₂ map FIG. 7(b) versus the e-CAMP T₂ map FIG. 7(c), and the difference image FIG. 7(d). FIG. 7(e-i) show plots of the probability histogram distributions of error in CSF FIG. 7(e), CSF+GM FIG. 7(f), GM FIG. 7(g), GM+WM FIG. 7(h) and WM FIG. 7(i). The presence of CSF, with its much higher T₂ value, tends to introduce bias and error.

FIG. 8 is a Table showing: (a) NRMSE and SSIM between e-CAMP reconstruction T₂ map and ground truth T₂ map or fully sampled reconstruction T₂ map of all simulations and experiments and (b) e-CAMP, fully sampled MESE, fully sampled SE reconstruction mean T₂ values in different MnCl₂ concentrations.

FIG. 9, which is composed of FIGS. 9(a) to 9(e), shows an overview of the data formation and the e-CAMP algorithm. Multi-echo images shown in FIG. 9(a) lead to the corresponding fully-sampled multi-coil k-space shown in FIG. 9(b), which are band-sampled to TSE data shown in FIG. 9(c). The phase prior is applied by synthesizing virtual conjugate coils and exploiting the phase conjugacy shown in FIG. 9(d). For careful initialization, the e-CAMP algorithm starts with the central bands and expands the bands gradually to ensure convergence to the global minimum FIG. 9(e).

FIG. 10, which is composed of FIGS. 10(a) to 10(c), shows projected Gradient Descent is illustrated. Projected gradient descent is an alternate strategy for minimizing the objective function. FIG. 10(a) visualizes the iteration with PGD. At the first stage, gradient descent updates the multi-echo image series without regard for the manifold constraint. Then the variables are projected onto the manifold to enforce the constraint, where the T₂ map is updated and the image series are aligned to the T₂ decay model, but the objective term increases. The interleaved pattern of the objective is shown in FIG. 10(b), with the images shown in FIG. 10(c).

FIG. 11, which is composed of FIGS. 11(a) to 11(l), includes FIGS. 11(a-d) that show that e-CAMP carefully handles the band-sampled pattern of TSE data while the existing methods fail. The non-expanding strategy (CAMP) also cannot ensure convergence to the global minimum. FIGS. 11(e-h) prove the advantage of virtual conjugate coils for the utilization of phase prior. The robust performance against parameter tuning is displayed in Figures (i-l).

FIG. 12, which is composed of FIGS. 12(a) to 12(e), shows reconstruction of retrospectively undersampled data using PGD, which is a method for minimizing the objective function that does not require significant parameter tuning. The T2 map of e-CAMP reconstruction shown in FIG. 12(b) achieves similar results to the ground truth shown in FIG. 12(a), especially in grey and white matter as shown in error map FIG. 12(d), error map excluding CSF shown in FIG. 12(e), and the one that emphasizes the grey matter and the white matter with an adjusted colorbar shown in FIG. 12(f). The regression plot shown in FIG. 12(c) quantifies the consistency of the reconstructed T2 map and the ground truth. The multi-echo images of e-CAMP reconstruction shown in FIG. 12(h) also attain similar results to the ground truth shown in FIG. 12(g), and the error map of FIG. 12(i) shows strong consistency in the central echoes (central bands) and weaker consistency in the edge echoes (edge bands).

FIG. 13, which is composed of FIGS. 13(a) to 13(e), shows reconstruction of T₂w-TSE data. FIGS. 13(a-e) show the case of a shorter echo train and FIGS. 13(f-j) show that of a longer echo train. The colorbar of the error maps (e&j) is adjusted to emphasize the error of the grey matter and the white matter. Both achieve acceptable results as the NRMSE is 10.6% and 14.7% in the whole brain and 8.9% and 12.3% in the white and grey matter, respectively. Note that the CSF region represents the major discrepancy. The second acquisition setting is more practical in clinical scans.

FIG. 14 is a schematic of an MRI system integrating the imaging process of the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

As discussed above, quantitative MRI (qMRI) requires specialized sequences, which has limited its adoption into routine clinical practice. On a national scale, a very small fraction of MRI exams includes a T₂ mapping scan, while nearly all include some kind of T₂w image scan. The present method and system, which is also referred to herein as e-CAMP (expanding-Constrained Alternating Minimization for Parameter mapping), overcomes this issue and calculates T₂ maps using only data from the T₂w image scan. Calculating T₂ maps directly from the clinical T₂w image data makes it much easier to collect large numbers of standardized images (i.e., images suitable for machine learning) from routine clinical practice.

In addition, previous research from other groups has shown that quantitative MRI (qMRI) is minimally affected by hardware and protocol settings. The problem with qMRI is that it comes from scans that are not routinely acquired. So getting a large number of them to train an AI algorithm is difficult, and such an AI would have limited utility. The present method and system provides a way to calculate qMRI from routine clinical MRI scan data.

As the following demonstrates, the present method and system takes on the highly challenging task of modeling scan data from a typical MR scan as if it was data from a (diabolically undersampled) qMRI scan. To make this feasible, the present invention introduces a novel cost function which converts the exponential model into a linear constraint, and the present invention also develops an initialization that gradually grows the data being considered. However, the novelty of the present invention goes beyond the specific implementation disclosed herein. The result of the present invention is that hardware and protocol settings are removed on the image, and the image is thus standardized.

The method associated with the implantation of e-CAMP does not require additional scans or any kind of training data. It can standardize individual images without requiring additional images or any extra information, and is guaranteed to keep all biological variability. It is also quite accurate.

The method and system of the present invention allows the underlying algorithm associated with e-CAMP to be added on to the software on the MRI machine, providing a bonus qMRI map alongside each standard and unmodified regular MR image. It could alternately be marketed as a standalone package to convert MR data into standardized qMRI maps, providing standardized MR images. Finally, it could be used as a competitive advantage in software providing some AI tasks. As such, and as those skilled in the art will appreciate the present invention is implemented via a computer, for example, operating with an Intel Core i7-8700 CPU and 32 GB RAM, and processing times are relatively quick.

As will be explained below in greater detail, e-CAMP standardizes the T₂-weighted images from TSE scans. Converting the exponential T₂-decay to a linear model, e-CAMP forms a biconvex minimization problem of data fidelity and T₂-decay model coherence. In addition, it adopts a strategy of gradually expanding the acquired k-space data into the reconstruction scope to avoid spurious local minima.

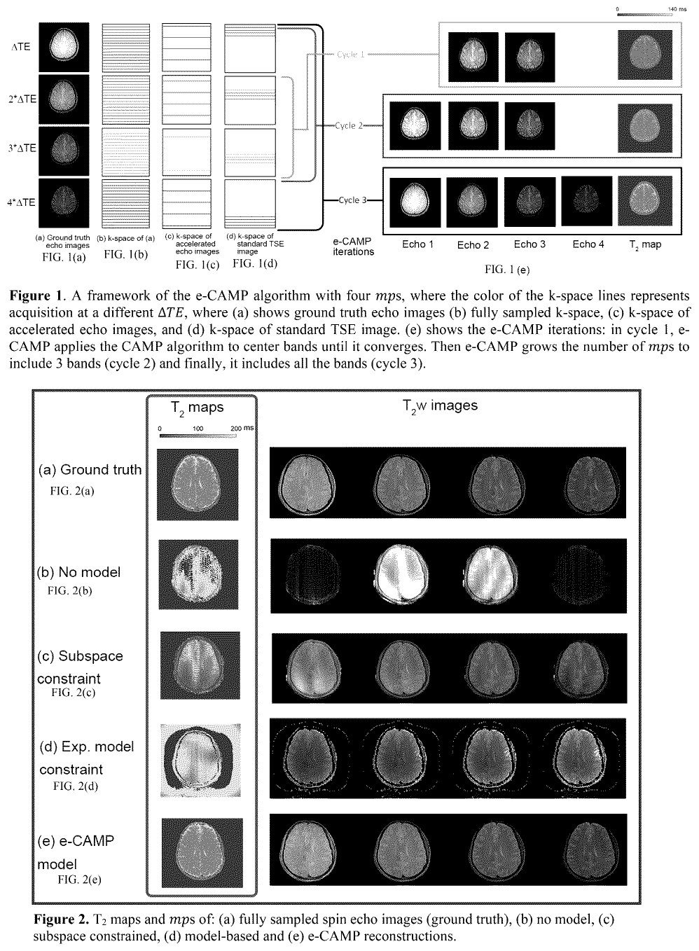

As explained below in detail, the present invention substantially modifies an algorithm called CAMP which stands for Constrained Alternating Minimization for Parameter mapping, that was previously reported. N. Dispenza, G. Galiana, D. Peters, R. Constable, and H. Tagare, Accelerated R1 or R2 Mapping with Geometric Relationship Constrained Reconstruction Method. As discussed above, the modified algorithm is called expanding-Constrained Alternating Minimization for Parameter mapping (e-CAMP). The e-CAMP cost function is notable for using an exact but linear expression to capture the exponential decay model and for being biconvex so that it can be alternately minimized relative to the image series or the T₂ map. A key feature distinguishing CAMP from e-CAMP is that the latter applies a stepwise initialization process that avoids spurious local minima due to the significant gaps in k-space. e-CAMP is iterative, and the first iteration begins with the echoes that span the middle of k-space, generating a blurry estimate of these images and the map that relates them (FIG. 1(e)). Once these have converged, additional echo images, reaching further out in k-space, are added to the reconstruction. FIG. 2(e) shows a reconstruction of the same data, i.e., non-overlapping strips of k-space at each echo, using the e-CAMP algorithm. Because e-CAMP converts the nonlinear physics model into a linear constraint and uses a sophisticated initialization, e-CAMP converges reliably to a good reconstruction of the individual echo images and the T₂ map.

Results using e-CAMP show that the undersampled reconstruction problem can be solved accurately using classical constrained-optimization methods. Both simulations and experiments show that e-CAMP can reconstruct accurate T₂ maps acquired with different parameters, such as echo spacing or echo train length. e-CAMP T₂ maps are also directly generated from single-image T₂w scans supplied by the MRI manufacturer (Siemens). These have been demonstrated in both phantoms and the human brain, showing good correspondence to the fully sampled T₂ mapping experiments. In addition, these latter studies included the acquisition of gold-standard spin-echo T₂ maps, i.e. single echo per acquisition rather than an echo train.

Many physical parameters such as T₁, B₀, B₁, and diffusion will affect the TSE signal. A generalized echo modulation (EMC) based on Bloch simulation is necessary to reduce these effects and emphasize the T₂ decay. N. Ben-Eliezer, D. K. Sodickson, and K. T. Block, Rapid and accurate T2 mapping from multi-spin-echo data using Bloch-simulation-based reconstruction, Magn Reson Med, vol. 73, no. 2, pp. 809-17, February, 2015. It is shown that with appropriate correction by an EMC Bloch dictionary, e-CAMP T₂ maps show good agreement with gold standard results in both the phantom and the human brain. These results strongly support the idea that e-CAMP generates maps that reflect a ground truth physical variable, so they can be used to standardize images from variable acquisition settings. Furthermore, because the maps are based on processing a type of data acquired in nearly every clinical protocol, e-CAMP could be a powerful tool in the aggregation of datasets suitable for large-scale analysis methods.

### CAMP

While the present method and system focuses on quantitative T₂-mapping, the CAMP algorithm which preceded the present invention (that is, e-CAMP), is broadly applicable to other image series that are related by a signal evolution model. CAMP applies to a dataset that acquires k-space for images sampling different values of a parameter weighting related to signal evolution, e.g., different TEs. The images are related by a signal model characterized by one or several parameter maps, e.g., a T₂ map. CAMP alternates between optimizing the image series and the parameter map. However, it minimizes a single cost function that incorporates both (1) consistency between each parameter image and its k-space data and (2) adherence of the image series to the relaxometry model for each pixel. Thus, the CAMP objective function consists of three terms: the first is concerned with data fidelity, the second is regularization, and the third is imposing the model constraint.

Data Fidelity Term The complete MRI signal vector S(k) is regarded as a series of signal vectors Sₚ(k), each acquired at the p^{th} parameter weighting, e.g., the p^{th} TE. Each Sₚ(k) samples some k-space for the corresponding complex image mₚ, and these encodings are contained in a matrix Eₚ. There are a total of P images, where P is also the number of parameter weightings and signal vectors.

The encoding matrix Eₚ(k) used in parameter mapping includes the effects of gradient encodings and coil sensitivities. K. P. Pruessmann, M. Weiger, P. Börnert, and P. Boesiger, Advances in sensitivity encoding with arbitrary k-space trajectories, Magn Reson Med, vol. 46, no. 4, pp. 638-51, October, 2001; B. P. Sutton, D. C. Noll, and J. A. Fessler, Fast, iterative image reconstruction for MRI in the presence of 541 field inhomogeneities, IEEE Transactions on Medical Imaging, vol. 22, no. 2, pp. 178-188, 2003. This encoding matrix has complex entries with a potentially different and complementary set of encodings for each p. Thus, the noisy signal vectors can be written as

$${S}_{p} ( k ) = {E}_{p} ( k ) {m}_{p} ( x ) + ε , \qquad ( 1 )$$

where ε is the additive noise.

Accordingly, the data fidelity term J₁ measures the error between the acquired data sets and our current estimate of the images.

$${J}_{1} = {\sum }_{p = 1}^{P} {‖ {S}_{p} - {E}_{p} {m}_{p} ‖}^{2} = {\sum }_{p = 1}^{P} {( {S}_{p} - {E}_{p} {m}_{p} )}^{H} ( {S}_{p} - {E}_{p} {m}_{p} ) , \qquad ( 2 )$$

where ( )^{H} refers to the complex conjugate transpose of a matrix. The L_2 norm in equation (2) may be replaced by other suitable norms, such as the L_1 norm.

### Regularization Term

To minimize the effect of MR noise, the solution is regularized by adding TV regularization.

$${J}_{2} = τ {\sum }_{p = 1}^{P} TV ( {m}_{p} ) , \qquad ( 3 )$$

where TV(mₚ)=Σₓ₌₁^{N}|∇mₚ(x)|, | [symbol] | refers to the modulus, and N is the number of pixels. J₂ promotes the sharpness of edges while reducing the effect of noise, however it does introduce an additional weighting parameter T that must be chosen appropriately. Note that J₁+J₂ is a convex function of each mₚ. M. Lustig, D. Donoho, and J. M. Pauly, Sparse MRI: The application of compressed sensing for rapid MR imaging, Magnetic Resonance in Medicine, vol. 58, no. 6, pp. 1182-1195, 2007.

### The CAMP Constraint Term

The physical model relating echo images provides information sharing among the undersampled images. For an image series with proton density PD experiencing T₂ relaxation sampled at equally spaced TE, mₚ(x) is given by:

$${m}_{p} ( x ) = P D ( x ) exp ( \frac{- p Δ T E}{{T}_{2} ( x )} ) . \qquad ( 4 )$$

Similarly, mₚ₊₁(x) has the form:

$${m}_{p + 1} ( x ) = P D ( x ) exp ( \frac{- p Δ T E}{{T}_{2} ( x )} ) exp ( \frac{- Δ T E}{{T}_{2} ( x )} ) \qquad ( 5 )$$

and is related to mₚ(x) as follows:

$${m}_{p + 1} ( x ) = {m}_{p} ( x ) α ( x ) , \qquad ( 6 )$$

where

$$α ( x ) = {e}^{\frac{- Δ T E}{{T}_{2} ( x )}}$$

and 0≤α(x)≤1. Notably, α is a map related to the T₂ map via a simple transformation:

$${T}_{2} ( x ) = \frac{- Δ T E}{ln ( α ( x ) )} . \qquad ( 7 )$$

A given (fixed) α(x) imposes an affine constraint on the sequence of mₚ images.

We impose the constraints of (5) using a penalty term J₃:

$${J}_{3} = λ {\sum }_{x = 1}^{N} {\sum }_{p = 1}^{P - 1} {‖ {m}_{p + 1} ( x ) - α ( x ) {m}_{p} ( x ) ‖}^{2} , \qquad ( 8 )$$

where λ>0 scales the penalty.

In this penalty function approach to imposing the constraints, the scale λ of the penalty term is set low in the initial minimization iterations and then increases as the iterations proceed.

### The Objective Function

The overall objective function for CAMP is:

$$J = {J}_{1} + {J}_{2} + {J}_{3} . \qquad ( 9 )$$

The objective function J can be minimized by alternately minimizing with respect to mₚ (fixed α) and then with respect to α (fixed mₚ), keeping in mind that α is real and constrained to 0≤α(x)≤1.

For fixed α, the function J is convex in each mₚ, and we minimize it with respect to mₚ using a nonlinear conjugate-gradient method (Polak-Ribiere). E. Polak, and G. Ribiere, “Note sur la convergence de méthodes de directions conjuguées,” ESAIM: 545 Mathematical Modelling and Numerical Analysis—Modélisation Mathématique et Analyse Numérique, vol. 546 3, no. 1, pp. 35-43, 1969. Gradients of various terms that are required for the conjugate-gradient method are as follows:

$${∇}_{{m}_{p}} {J}_{1} = 2 {E}_{p}^{H} ( {S}_{p} - {E}_{p} {m}_{p} ) \qquad ( 10 )$$

$${∇}_{{m}_{ρ}} {J}_{2} = - τ ( \frac{\partial }{\partial {x}_{1}} \frac{{∇}_{x} {m}_{p} ( x )}{\vert {∇}_{x} {m}_{p} ( x ) \vert} + \frac{\partial }{\partial {x}_{2}} \frac{{∇}_{x} {m}_{p} ( x )}{\vert {∇}_{x} {m}_{p} ( x ) \vert} ) \qquad ( 11 )$$

$${∇}_{{m}_{p}} {J}_{3} = 2 λ ( α ( x ) ∘ α ( x ) ∘ {m}_{1} ( x ) - α ( x ) ∘ {m}_{2} ( x ) ) ,$$

$$when p = 1$$

$${∇}_{{m}_{p}} {J}_{3} = 2 λ ( {m}_{p} ( x ) + α ( x ) ∘ α ( x ) ∘ {m}_{p} ( x ) - α ( x ) ∘ ( {m}_{p - 1} ( x ) + {m}_{p + 1} ( x ) ) ,$$

$$where 1 < p < P$$

$${∇}_{{m}_{p}} {J}_{3} = 2 λ ( {m}_{p} ( x ) - α ( x ) ∘ {m}_{p - 1} ( x ) ) , \qquad ( 12 )$$

$$when p = P ,$$

where ° is the Hadamard product.

Only J₃ is relevant when minimizing J with respect to α(x). Note that J₃ is a sum of spatially independent terms, each term of the type Σₚ₌₁^{P-1}∥mₚ₊₁(x)−α(x)mₚ(x)∥², and these terms are uncoupled as far as α(x) is concerned. Thus, to minimize J₃ we can minimize each term independently with respect to α(x). This minimization requires some care since α(x) is required to be real, while the set of mₚ images is complex. Furthermore, α(x) is required to satisfy 0≤α(x)≤1. We deal with this in two steps. First, the real-valued nature of α(x) is handled by allowing α(x) to be complex while imposing the constraint that the imaginary entries of α(x) be equal to zero. In the second step, α(x) is constrained to be between 0 and 1.

The α(x) that minimizes the term Σₚ₌₁^{P-1}∥mₚ₊₁(x)−α(x)mₚ(x)∥² has a closed-form solution, which is easily expressed in matrix notation. We denote the real and imaginary parts of mₚ(x), α(x) by subscripts r and i:

$$\begin{matrix}{m}_{p} ( x ) = {m}_{p r} ( x ) + j {m}_{p i} ( x ) , where 1 \leq p \leq P , \\ α ( x ) = {α}_{r} ( x ) + j {α}_{i} ( x )\end{matrix}$$

Next, the real and imaginary parts of these are used to define the following:

$$\begin{matrix}( x ) = [ \begin{matrix}{α}_{r} ( x ) \\ {α}_{i} ( x )\end{matrix} ] & A ( x ) = [ \begin{matrix}{m}_{1} ( x ) \\ ⋮ \\ {m}_{P - 1} ( x )\end{matrix} ] , & Y ( x ) =\end{matrix} [ \begin{matrix}{m}_{2 r} ( x ) \\ ⋮ \\ {m}_{Pr} ( x ) \\ {m}_{2 i} ( x ) \\ ⋮ \\ {m}_{Pi} ( x )\end{matrix} ] .$$

### Taking

$$H ( x ) = [ \begin{matrix}{A}_{r} ( x ) & - {A}_{i} ( x ) \\ {A}_{i} ( x ) & {A}_{r} ( x )\end{matrix} ]$$

to be a matrix made out of the real and imaginary parts of A(x), it is a straightforward matrix calculation to show that (Y(x)−H(x)X(x))^{T} (Y(x)−H(x)X(x))=Σₚ₌₁^{P-1}∥mₚ₊₁(x)−α(x)mₚ(x)∥², which is the term we wish to minimize. Finally, requiring the imaginary part of α(x) to be zero can be expressed as a constraint on X(x) by defining

$$w = [ \begin{matrix}0 \\ 1\end{matrix} ]$$

and requiring w^{T}X(x)=0.

Thus, minimizing each term in J₃ corresponds to minimizing (Y(x)−H(x)X(x))^{T} (Y(x)−H(x)X(x)) with respect to X(x) subject to the constraint w^{T}X(x)=0. We solve the constrained minimization by constructing the Lagrangian, (Y(x)−H(x)X(x))^{T} (Y(x)−H(x)X(x))+2κ w^{T}X(x), where κ is the Lagrange multiplier, and find the unconstrained minimum of the Lagrangian. The unconstrained minimum is given by:

$$( x ) = [ \begin{matrix}{α}_{r} ( x ) \\ {α}_{i} ( x )\end{matrix} ] = {( {H}^{T} ( x ) H ( x ) )}^{- 1} ( {H}^{T} ( x ) Y ( x ) - κ w ) \qquad ( 13 )$$

The Lagrangian multiplier κ is found by imposing the constraint w^{T}X(x)=0:

$$κ = \frac{{{w}^{T} ( {H}^{T} ( x ) H ( x ) )}^{- 1} H ( x ) Y ( x )}{{{w}^{T} ( {H}^{T} ( x ) H ( x ) )}^{- 1} w} \qquad ( 14 )$$

$$Finally , we set α ( x ) = min ( max ( {α}_{r ( x )} , 0 ) , 1 ) \qquad ( 15 )$$

To summarize, CAMP reconstructs the complete data Sₚ by applying the following iterative procedure. Superscripts in this procedure refer to the iteration number, and the constraint penalty weight is increased in the following step (3.c) to impose the enforcement of the constraint.
1. Initialize a set of m⁰ₚ by solving

$${min}_{{m}_{p}} {\sum }_{p = 1}^{P} {‖ {S}_{p} - {E}_{p} {m}_{p}^{0} ‖}^{2}$$

using conjugate gradients.
2. Initialize α⁰ from mₚ⁰ using closed-form expression as described in (13)-(15).
3. For iteration l:
   a. Fixing α to α^{l-1} minimizes J with respect to mₚ using conjugate gradients via the Polak-Ribiere algorithm. E. Polak, and G. Ribiere, Note sur la convergence de méthodes de directions conjuguées, ESAIM: Mathematical Modelling and Numerical Analysis—Modélisation Mathématique et Analyse Numérique, vol. 3, no. 1, pp. 35-43, 1969. The minimized set of mₚ images is then identified by mₚ^{l}.
   b. Fixing all mₚ to mₚ^{l}, minimizes J with respect to α as described in (13)-(15). Set α¹ to the minimizing values.
   c. Increase λ to enforce the constraint more tightly.
   d. Repeat a-c until the relative fractional change in the sum of all the mₚ images becomes smaller than a threshold Eₚ₌₁^{P}=∥mₚ^{l+1}−mₚ^{l}∥/∥mₚ^{l}∥<ε.
      e-CAMP

The general formation of e-CAMP is recapitulated as follows. A clinical T₂w imaging pulse sequence (T₂w-TSE) acquires a band of k-space at each echo time, as illustrated in FIG. 1(a-c). We define the p-th underlying image ρₚ∈C^{N} within a total number of P images in the series, where P is the echo train length (ETL) and N is the image size. The entire image series is referred to as ρ={ρ₁, . . . , ρ_{P}}. Note that the length of the image series equals ETL. The acquired k-space data s_{p,c}∈C^{m} is a band of the full k-space data sₑ∈C^{M}, where coil index c=1, . . . , C. M and m are the full k-space and k-space band size, respectively.

Each coil sensitivity map [symbol] ∈C^{N} is vectorized to a diagonal matrix and stacked vertically to perform parallel imaging.

$$\\ \begin{matrix}C = ( \begin{matrix}diag ( {\hat{C}}_{1} ) \\ ⋮ \\ diag ( {\hat{C}}_{C} )\end{matrix} ) & ( 16 )\end{matrix}$$

For each echo, the sampling mask \hat{M}∈R^{M} is defined as 1 in the sampling region and 0 in the rest. It is vectorized to a diagonal matrix, with m non-zero rows extracted from the total M rows. It is then replicated C times and stacked vertically to form Mₚ∈R^{Cm×M} so that sampling is identical for all coils.

$$\\ \begin{matrix}{M}_{p} = ( \begin{matrix}{\underbrace{diag ( \hat{M} )}}_{\begin{matrix}Extract \\ non ‐ zero rows\end{matrix}} \\ ⋮ \\ {\underbrace{diag ( \hat{M} )}}_{\begin{matrix}Extract \\ non ‐ zero rows\end{matrix}}\end{matrix} ) & ( 17 )\end{matrix}$$

To preserve matrix multiplication, Fourier transform \hat{F}∈C^{M×N} is replicated and stacked diagonally to yield a block-diagonal matrix F∈C^{CM×CN}.

$$\\ \begin{matrix}F = ( \begin{matrix}\hat{F} & 0 & ⋯ & 0 \\ 0 & \hat{F} & ⋯ & 0 \\ ⋮ & ⋮ & ⋱ & ⋮ \\ 0 & 0 & ⋯ & \hat{F}\end{matrix} ) & ( 18 )\end{matrix}$$

Taking the Gaussian noise εₚ∈C^{Cm} into consideration, the forward model is formulated as

$$\\ \begin{matrix}\begin{matrix}{s}_{p} = {M}_{p} FC {ρ}_{p} + {ϵ}_{p} \\ = {E}_{p} {ρ}_{p} + {ϵ}_{p} ,\end{matrix} & ( 19 )\end{matrix}$$

$$\\ p = 1 , … , P$$

The transformation is summarized as the encoding matrix Eₚ∈C^{Cm×N}.
In addition to the data fidelity term, a total variation (TV) regularization term is applied to the objective function to ensure the piecewise smooth nature of an image.

$$\\ \begin{matrix}TV ( {ρ}_{p} ) = {\sum }_{x , y} \sqrt{{\vert {ρ}_{p} ( x + 1 , y ) - {ρ}_{p} ( x , y ) \vert}^{2} + {\vert {ρ}_{p} ( x , y + 1 ) - {ρ}_{p} ( x , y ) \vert}^{2}} , & ( 20 )\end{matrix}$$

$$\\ p = 1 , … , P$$

where ρₚ is the original 2D version of the vectorized image ρₚ. Multi-echo data conforms to the signal evolution model of exponential T₂ decay, determined by the T₂ map T₂∈R^{N}. The exponential decay model is transformed into a linear expression [Elsaid Nahla M. H., Tagare Hemant D., Galiana Gigi. A Physics-Based Algorithm to Universally Standardize Routinely Obtained Clinical T₂-Weighted Images. Academic Radiology. 2024; 31(2):582-595, which is incorporated herein by reference] by creating a new variable α(x)=exp (−ΔTE/T₂ (x)), 0<α(x)<1, α∈R^{N}, and ΔTE is the echo spacing (ES).

$$\\ \begin{matrix}{ρ}_{p + 1} = α {ρ}_{p} , p = 1 , … , P - 1 & ( 21 )\end{matrix}$$

In conclusion, the constrained minimization problem is formulated as

$$\\ \begin{matrix}{{min}_{ρ , α}}_{} {\sum }_{p = 1}^{P} [ {‖ {s}_{p} - {E}_{p} {ρ}_{p} ‖}^{2} + λ TV ( {ρ}_{p} ) ] s . t . {ρ}_{p + 1} = α {ρ}_{p} , p = 1 , … , P - 1 & ( 22 )\end{matrix}$$

The previously described embodiment of e-CAMP enforced the constraint of Equation (22) by adding a corresponding penalty term to the objective function. The objective function was then minimized in an unconstrained fashion.

Given that the constrained optimization is non-convex, careful initialization is needed to ensure convergence to the global minimum. Although initialization based on zero-filled k-space is common, doing so with band-sampled Cartesian data leads to an image series that is significantly far away from the constraint and causes convergence to a spurious local minimum. e-CAMP addresses this issue by first reconstructing central echo images for which bands close to the center of k-space were acquired. This problem is allowed to converge, satisfying the constraint, and the result serves as initialization for subsequent iterations. In subsequent iterations, additional bands are recruited into the optimization problem in an expanding fashion. Note that while the above-mentioned process is tailored for T₂ mapping, e-CAMP can be modified for other quantitative mapping scenarios with a signal evolution model.

The algorithm and its initialization can also be augmented by the addition of a prior which relates voxel values in T₂w images with corresponding voxel values in T₂ images. This prior may take different forms for different tissue classes.

While CAMP is applicable to any encoding strategy, including TSE, the uneven sampling in the k-space bands initializes strong intensity biases between the images, which can be difficult to overcome. e-CAMP mitigates this by first applying CAMP only to the center k-space bands for a few iterations. These iterations reconstruct the center two mₚ images and an initial estimate of alpha. Subsequent iterations progressively include bands towards the edge of the k-space. Each cycle introduces an additional mₚ per k-space band, as illustrated in FIG. 1.

The details of the above are as follows:

The center bands are initialized using a scale factor derived from the ratio of their l² norms. Then any newly added mₚ is initialized using the derived α(x) from the previous cycle as well as the neighboring band. In addition, in some embodiments, the phase of each my is assumed to be that of the standard T₂w image that results from treating all data as part of a single k-space. Accordingly, we incorporated that phase into Eₚ and forced each update to the mₚ series to have real values. This is referred to as strict enforcement of the phase prior.

### Simulations

Simulations were carried out using the proton density (PD) and T₂ maps extracted from previously acquired fully sampled data. Coil weightings were derived from experimental coil profiles in an eight-channel coil. Data were simulated to mimic a T₂w acquisition during monoexponential decay in each voxel, where k-space is acquired band-wise across eight echoes. Different patterns of acquisitions were simulated at echo spacings of 20 ms, 25 ms, 30 ms, and 35 ms, with the center of k-space acquired at the fifth echo. Random Gaussian noise with zero mean and a standard deviation of 5% of the l²-norm of the signal was added to the data.

The quality of the reconstructed map using the simulated undersampled k-space was compared to the reference map ground truth using normalized root mean square error (NRMSE).

In reality, partial volume effects are likely to compromise the monoexponential assumption in e-CAMP. Therefore, to assess the sensitivity of the reconstruction to partial volume, we carried out another simulation using PD and T₂ values taken from the literature. J. Warntjes, O. Leinhard, J. West, and P. Lundberg, Rapid magnetic resonance quantification on the brain: Optimization for clinical usage, Magnetic Resonance in Medicine vol. 60, no. 2, pp. 320-329, 2008. For this partial volume simulation, a T₁w image was segmented into five compartments: Cerebrospinal Fluid (CSF), CSF+gray matter (GM), GM, GM+white matter (WM), and WM. The segmentation maps were used to generate eight echo TSE data exhibiting 50/50 biexponential decay in the mixed compartments. e-CAMP, which is based on a monoexponential model of signal decay, was used for reconstruction. Random Gaussian noise of zero mean and 5% standard deviation of the l²-norm of the signal was added to the data.

### Retrospective Human Brain Experiments

All participants provided written informed consent according to procedures approved by our Institution's IRB.

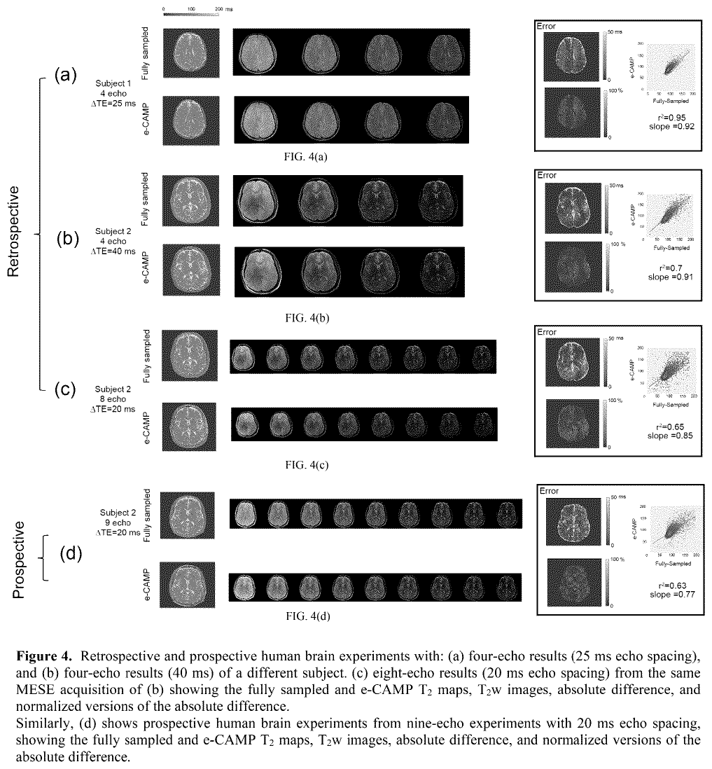

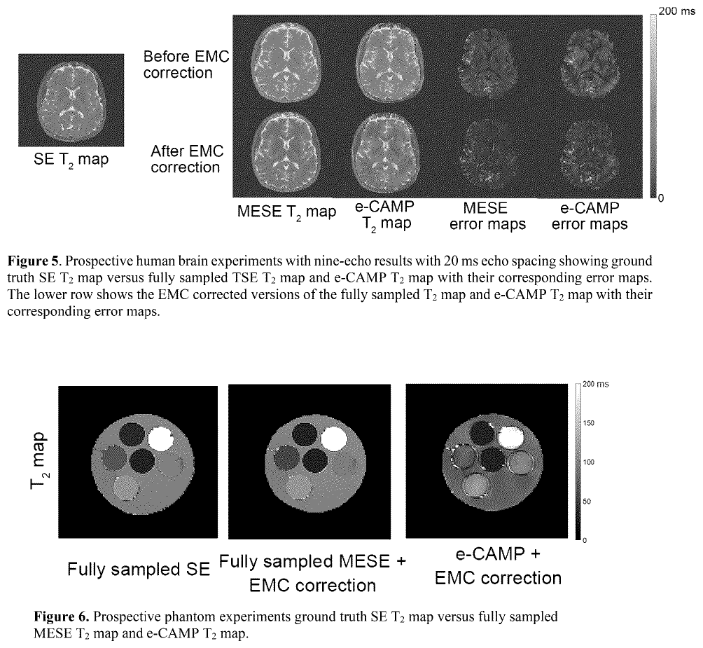

Cartesian single Spin-Echo (SE) images were acquired on a 3 T MRI scanner (TIM Trio, Siemens Healthcare, Erlangen, Germany) using a 12-channel RF head coil in a circularly polarized mode so that four channels were acquired with combinations of the coil elements. Here, data were acquired using spin-echo sequences with TR=2500 ms, TE=25, 50, 75, and 100 ms, BW=280 Hz/pix, 250 mm² field-of-view with a base resolution of 128, 3 mm slice thickness, and acquisition time=320 s. Fully sampled k-space was acquired at each TE and retrospectively undersampled (FIG. 4a).

In addition, Cartesian 8-echo images were acquired on a 3 T MRI scanner (MAGNETOM Prisma^{fit}; Siemens Healthcare, Erlangen, Germany) using a 20-channel RF head coil. In this experiment, T₂w images were acquired using a Multi-Echo Spin-Echo (MESE) sequence with TR=3000 ms, TE=20, 40, 60, 80, 100 120, 140 160 ms, BW=480 Hz/pix, 220 mm² field-of-view with a resolution of 128 pixels, and 3 mm slice thickness. These data were acquired with full k-space sampling at each echo, contrasts=8, and then retrospectively reconstructed with echo spacing of 40 ms (excluding odd echoes) as illustrated in FIG. 4b. Additionally, the same data was retrospectively reconstructed with the full eight contrasts with 20 ms echo spacing, as shown in FIG. 4c.

### Prospective Human Brain Experiments

e-CAMP was used to reconstruct the T₂ map using a T₂w image that was acquired using the TSE Siemens product sequence (ETL=9, echo spacing=20 ms). TR=3000 ms, BW 219 Hz/pix, 220 mm² field-of-view with a resolution of 128 pixels, and 3 mm slice thickness. For validation, we acquired fully sampled TSE images and fully sampled spin echo images.

The acquisition parameters of the fully sampled MESE were TR=3000 ms, 20, 40, 60, 80, 100 120, 140 160 ms, BW 219 Hz/pix, 220 mm² field-of-view with a resolution of 128 pixels, and 3 mm slice thickness.

The acquisition parameters of the SE images were TR=3000 ms, TE=40, 80, 120, 160 ms, BW 219 Hz/pix, 220 mm² field-of-view with a resolution of 128 pixels, and 3 mm slice thickness.

To compare the TSE reconstructions to the SE reconstructions, we used the echo modulation curve (EMC) corrections to be applied to the T₂w images reconstructed using TSE image acquisitions. N. Ben-Eliezer, D. K. Sodickson, and K. T. Block, “Rapid and accurate T2 mapping from multi-spin-echo data using Bloch-simulation-based reconstruction,” Magn Reson Med, vol. 73, no. 2, pp. 809-17, February, 2015. Inputting the nominal flip angles of the scan and a list of uniform T₁ of 1000, 1500, 3000 ms, a dictionary of echo modulation curves was generated for T₂ values between 1 and 300 ms. Using the echo train image series (either generated by e-CAMP or reconstruction of full k-space), the intensity of a pixel across echo times was matched to an entry in the dictionary. The T₂ corresponding to the closest matching entry was taken to be the gold standard T₂ of that pixel.

### Phantom Experiments

A phantom was built in-house using six tubes filled with varying concentrations of MnCl₂ solutions immersed in an agar gel solution to gradually increase MRI signal intensity.

Phantom Cartesian spin-echo images were acquired on a 3 T MRI scanner (MAGNETOM Prisma^{fit}; Siemens Healthcare, Erlangen, Germany) using a 20-channel RF head coil. In addition, during the same scanning exam, a set of fully sampled Cartesian 10-echo images were acquired using a MESE sequence. Finally, a T₂w image was acquired using a Siemens product TSE sequence (ETL=9, echo spacing=13 ms). TR=3000 ms, BW 219 Hz/pix, 180 mm² field-of-view with a resolution of 128 pixels and 5 mm slice thickness. The acquisition parameters of the spin echo and the fully sampled MESE were TR=3000 ms, 20, 40, 60, 80, 100 120, 140 160 ms, BW 219 Hz/pix, 180 mm² field-of-view with a resolution of 128 pixels, and 5 mm slice thickness. Once again, the echo modulation curve (EMC) corrections were applied to the T₂w images that were reconstructed using TSE image acquisitions, as shown in FIG. 5.

### Reconstruction

With GPU calculations enabled, calculations were performed in MATLAB (MathWorks Inc, Natick, Massachusetts, USA).

From the 8-echo retrospective human brain experiments, two retrospective TSE versions were created: one with ΔTE=40 ms using the k-space lines from images with TE=20, 60, 100, 160 ms, and the other with ΔTE=20 ms, using the k-space bands from the entire eight contrasts. In both cases, the central k-space was acquired at a TE of 100 ms. Separately measured complex sensitivity profiles were incorporated into the encoding matrix.

The data of 4-echo retrospective human brain experiments were undersampled to complementary bands with central k-space acquired at TE=75 ms.

In CAMP, the penalty weight λ was initialized by a value that maintains the value of J₃ to be on the order of magnitude of that of J₁ and then increased by 15% at each iteration. However, in e-CAMP, it is desirable to emphasize data fidelity during the first cycle that uses the central bands of k-space. Therefore, λ was used with an order of magnitude less than the rest of the iterations in that first cycle. Similarly, the TV-weight τ was chosen to maintain the value of J₂ to be on the order of magnitude of J₁ (τ˜0.001). After extensive numerical experimentation, these values were found to give stable convergence. The initial m⁰ was reconstructed with 10 CG iterations. Approximately ten iterations suffice to minimize J with respect to mₚ and 10-15 alternating minimizations over α, and the mₚ images were sufficient to achieve overall convergence.

Subspace-constrained and model-based reconstructions were implemented with the Berkeley Advanced Reconstruction Toolbox (BART). M. Uecker, F. Ong, J. I. Tamir, D. Bahri, P. Virtue, J. Y. Cheng, T. Zhang, and M. Lustig, Berkeley Advanced Reconstruction Toolbox. For subspace-constrained reconstruction, a dictionary simulating 1000 T₂ values between 20-1000 ms was generated and used to identify 2 basis functions for 4 echo acquisitions (data of FIG. 2) and 4 basis functions for 9 echo acquisitions (data of FIG. 6 and Supplementary Information). Reconstructions studying the performance of interleaved reconstruction patterns used evenly spaced retrospective undersampling of the fully sampled echo train data. Reconstruction was performed with 100 iterations and no additional regularization. The image series resulting from reconstruction was then fit to an exponential to identify the T₂ of each pixel. For model-based reconstruction, data were first coil-compressed to 8-channel data, followed by reconstruction with L1 regularization, 100 inner iterations, and 8 Newton steps. R₂ maps were scaled by 10, as appropriate for this output, before conversion to T2 values.

The quality of the reconstructed map using the undersampled k-space was assessed using root mean square error (NRMSE) and SSIM. W. Zhou, A. C. Bovik, H. R. Sheikh, and E. P. Simoncelli, “Image quality assessment: from error visibility to structural similarity,” IEEE Transactions on Image Processing, vol. 13, no. 4, pp. 600-612, 2004. The NRMSE between the undersampled reconstructed map \tilde{Y} and the fully sampled reconstructed map Y are calculated using the following equation, as often defined in quantitative reconstruction literature [M. Lustig, and J. M. Pauly, SPIRiT: Iterative self-consistent parallel imaging reconstruction from arbitrary k-space, Magn Reson Med, vol. 64, no. 2, pp. 457-71, August, 2010; J. Zou, J. M. Balter, and Y. Cao, Estimation of pharmacokinetic parameters from DCE-MRI by extracting long and short time-dependent features using an LSTM network, Med Phys, vol. 47, no. 8, pp. 3447-3457, August, 2020; J. Duan, Y. Liu, and P. Jing, Efficient operator splitting algorithm for joint sparsity-regularized SPIRiT-based parallel MR imaging reconstruction, Magnetic Resonance Imaging, vol. 46, pp. 81-89, 2018 Feb. 1, 560 2018.]:

$$\\ \begin{matrix}NRMSE = \frac{{[ \frac{{\sum }_{x = 1}^{N} {( {Y}_{x} - {\tilde{Y}}_{x} )}^{2}}{N} ]}^{\frac{1}{2}}}{max ( Y ) - min ( Y )} . & ( 23 )\end{matrix}$$

### Results

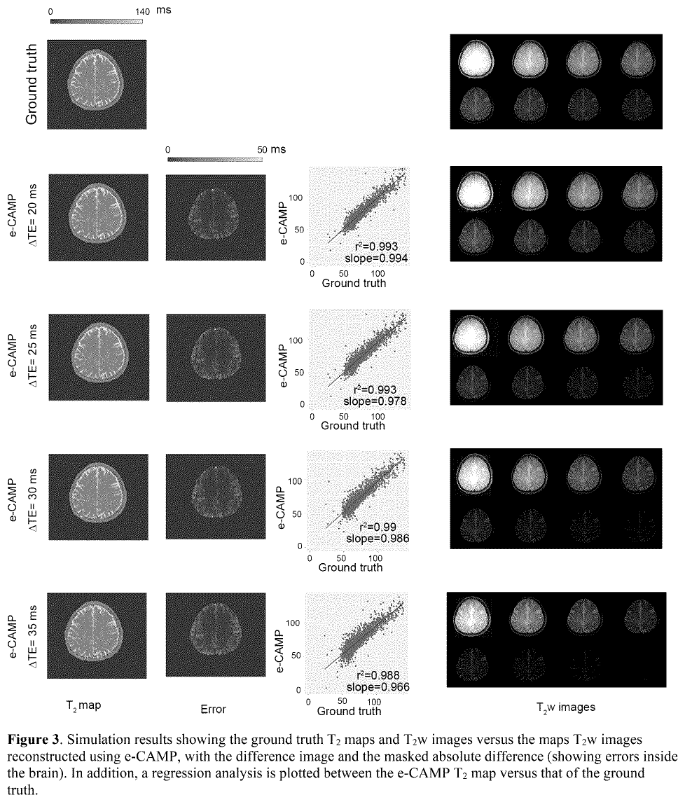

e-CAMP was first tested in simulation studies to mimic various acquisition parameters. The top row of FIG. 3 shows the ground truth T₂ map used for simulations and the images associated with each echo time for a spacing of 20 ms. Below this is the e-CAMP T₂ map reconstructed from the band-sampled k-space of each echo image (i.e., a pattern similar to FIG. 1(d)). Subsequent columns show deviation from the ground truth T₂, a scatter plot of T₂ values and the associated image series from the e-CAMP reconstruction. Similar simulations performed for echo spacings of 25, 30, and 35 ms give highly consistent and accurate maps and image series. In all cases, the slope is within 5% of 1, and the coefficient of determination is r2>0.96. The e-CAMP T₂ maps are also highly consistent across echo spacings, with an SSIM>0.98 across all pairs.

FIG. 4 shows e-CAMP results from experimentally acquired data with both retrospective undersampleing and true vendor T₂w acquisitions. FIG. 4(a) shows the results from a study where four single echo acquisitions were obtained with full sampling at each echo time. The first row of FIG. 4(a) shows the reference T₂ map and images associated with fitting fully sampled images to a monoexponential. Below this, are the T₂ maps and echo images from e-CAMP reconstruction after retrospective band-undersampling of these data. Plots to the right show more detailed analyses of error in the e-CAMP T₂ maps, namely, absolute error in both milliseconds and %.

FIG. 4(b) shows the results from a different subject where eight echo times are collected in a consecutive echo train, with full k-space acquisition at each echo. The first row shows the reference T₂ map and images generated from fully sampled k-space, using only even echoes (i.e., 40 ms echo spacing). Below these are the e-CAMP reconstruction with the even echoes, simulating a longer echo spacing (40 ms) with 32 k-space lines per band.

FIG. 4(c) shows the reconstructions of the same data of FIG. 4(b), but this time with the full range of echoes (i.e., eight echoes and 20 ms echo spacing). The maps again show excellent agreement with the fully sampled case, particularly in regions of brain tissue that do not border CSF. Notably, the T₂ maps made from data with different echo spacing and echo train length (FIGS. 4(b) and 4(c)) show excellent agreement (SSIM=0.92), comparable to the similarity seen in simulations (SSIM>0.98).

FIG. 4(d) shows e-CAMP results using actual T₂w image data from a Siemens TSE product sequence, i.e., with echo train acquisition and prospective undersampling of the k-space at each echo time. While product sequences implement finer and symmetric band sampling, these results demonstrate the feasibility of generating a quantitative T₂ map directly from clinical T₂w image data. R. T. Constable, and J. C. Gore, The Loss of Small Objects in Variable TE Imaging: Implications for FSE, RARE, and EPI, Magnetic Resonance in Medicine, vol. 28, no. 1, pp. 9-24, 1992.

It is well known that the signal decay observed in an echo train does not only reflect pure T₂ decay, primarily due to contributions from stimulated echoes. While conventional clinical T₂ mapping accepts this discrepancy, we also aimed to study whether Bloch-based corrections could be applied to reconstruct a gold standard T₂ as measured by a series of single spin echo experiments. Gold standard T₂ maps would provide a maximally reproducible and site-independent standardization.

On the same subject of FIG. 4(d), a series of fully sampled SE images was used to compute a gold standard T₂ map, shown on the left of FIG. 5. T₂ maps generated from fully sampled echo train images and a T₂ map generated from the e-CAMP reconstruction of T₂w data are shown in the first row, with error maps showing absolute deviation relative to the gold standard T₂ map. As expected, the different physics of single echo vs. echo train acquisition led to systematic deviations from the gold standard T₂ values, although these deviations are comparable in both the fully sampled and e-CAMP reconstructions.

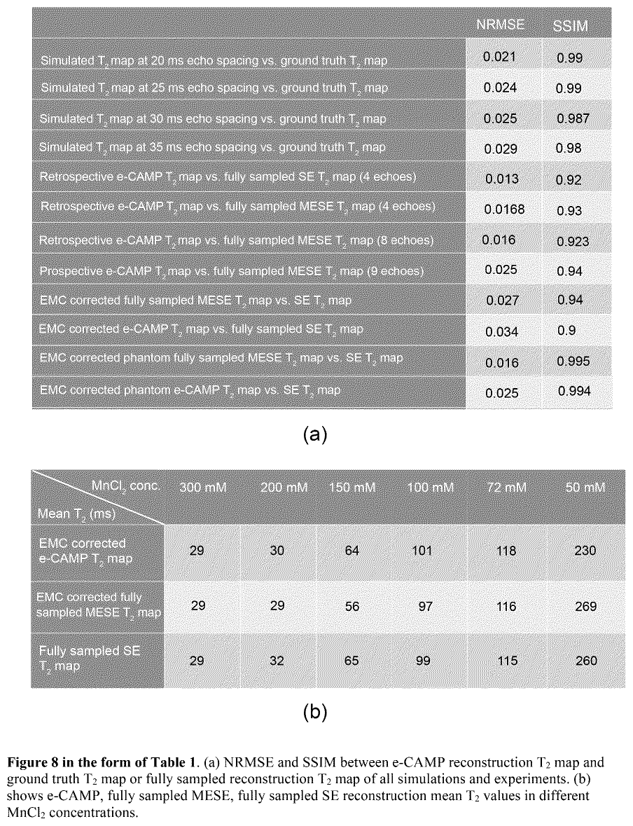

To achieve better agreement with gold standard values, a previously published T₂ correction based on Bloch simulations of a realistic spin echo train was tested. N. Ben-Eliezer, D. K. Sodickson, and K. T. Block, Rapid and accurate T₂ mapping from multi-spin-echo data using Bloch-simulation-based reconstruction, Magn Reson Med, vol. 73, no. 2, pp. 809-17, February, 2015. The second row of FIG. 5 shows Bloch-corrected T₂ maps from both fully sampled and e-CAMP reconstructions. The bias in images is clearly reduced, and the absolute error is reduced by 1.2% in NRMSE (see FIG. 8—Table 1a). While this approach does not represent a state-of-the-art correction between echo train and gold standard T₂ values, having been performed without a B0 or B1 map, the results show the feasibility of generating T₂ maps in good agreement with gold standard methods directly from the data in T₂w image acquisition.

A similar study was also performed on a MnCl₂ T₂ phantom. FIG. 6 shows T₂ maps generated from gold standard spin echo experiments, a fully sampled echo train experiment, and a product T₂w imaging experiment reconstructed with e-CAMP. The latter studies were corrected with a Bloch-derived dictionary to account for discrepancies between single echo and multiecho signal evolution. As before, following Bloch-derived correction of the T₂ values, both fully sampled and e-CAMP T₂ maps show excellent agreement with the gold standard result; see also the mean T₂ values in Table 1b of FIG. 8. As discussed in the Supplementary Information, model-based reconstruction methods do not generate usable T₂ maps for this type of data, despite solid performance for other undersampled datasets. The average T₂ values in each vial are summarized in Table 1b of FIG. 8.

It should be appreciated that the motivation underlying the development of e-CAMP is different from previous studies reconstructing T₂ maps from undersampled data. Previous methods focus on generating T₂ maps from short scans, so they make use of the flexibility in k-space sampling to achieve high accuracy and acceleration from more distributed k-space sampling patterns. M. A. Cloos, F. Knoll, T. Zhao, K. T. Block, M. Bruno, G. C. Wiggins, and D. K. Sodickson, Multiparametric imaging with heterogeneous radiofrequency fields, Nature Communications, vol. 7, no. 1, pp. 12445, 2016/08/16, 2016; K. T. Block, M. Uecker, and J. Frahm, Model-based iterative reconstruction for radial fast spin-echo MRI, IEEE transactions on medical imaging, vol. 28, no. 11, pp. 1759-1769, 2009 November, 2009; T. J. Sumpf, A. Petrovic, M. Uecker, F. Knoll, and J. Frahm, Fast T₂ Mapping With Improved Accuracy Using Undersampled Spin-Echo MRI and Model-Based Reconstructions With a Generating Function, IEEE Transactions on Medical Imaging, vol. 33, no. 12, pp. 2213-2222, 2014; C. Huang, C. G. Graff, E. W. Clarkson, A. Bilgin, and M. I. Altbach, T₂ mapping from highly undersampled data by reconstruction of principal component coefficient maps using compressed sensing, Magn Reson Med, vol. 67, no. 5, pp. 1355-66, May, 2012; G. Nataraj, J. F. Nielsen, C. Scott, and J. A. Fessler, Dictionary-Free MRI PERK: Parameter Estimation via Regression with Kernels, IEEE Trans Med Imaging, vol. 37, no. 9, pp. 2103-2114, September, 2018; F. H. Petzschner, I. P. Ponce, M. Blaimer, P. M. Jakob, and F. A. Breuer, Fast MR parameter mapping using k-t principal component analysis, Magn Reson Med, vol. 66, no. 3, pp. 706-16, September, 2011; J. I. Tamir, M. Uecker, W. Chen, P. Lai, M. T. Alley, S. S. Vasanawala, and M. Lustig, T₂ shuffling: Sharp, multicontrast, volumetric fast spin-echo imaging, Magnetic Resonance in Medicine, vol. 77, no. 1, pp. 180-528 195, 2017; X. Wang, Z. Tan, N. Scholand, V. Roeloffs, and M. Uecker, Physics-based reconstruction methods for magnetic resonance imaging, Philosophical Transactions of the Royal Society A: Mathematical, Physical and Engineering Sciences, vol. 379, no. 2200, pp. 20200196, 2021; X. Wang, S. Rosenzweig, N. Scholand, H. C. M. Holme, and M. Uecker, Model-based reconstruction for simultaneous multi-slice mapping using single-shot inversion-recovery radial FLASH, Magnetic Resonance in Medicine, vol. 85, no. 3, pp. 1258-1271, 2021. Previous accelerated T₂ mapping methods have broken down when applied to the band-undersampling of standard clinical T₂w image data. This is because the increase in T₂ weighting across echoes is overshadowed by the energy differences associated with different parts of k-space, as further described in the Supplementary Information.

e-CAMP reconstructs a T₂ map without adding a dedicated scan to the protocol. e-CAMP is designed to accept the constraints of standard clinical T₂w k-space sampling. e-CAMP features that make this possible include (1) the linearized and exact form of the physics constraint, (2) the phase conjugacy property, and (3) the growing heuristic. e-CAMP is the first to successfully demonstrate that data from an unmodified T₂w scan can be used to generate a T₂ map.

The advantage of generating T₂ maps directly from T₂w imaging data is that these scans are already included in most clinical protocols. Therefore, there is no need to alter the clinical protocol, or the set of images reviewed by practicing clinicians, as these images can be generated on an auxiliary reconstruction pipeline. While e-CAMP does impose a relatively simple model and may not achieve the accuracy of dedicated state-of-the art T₂ mapping acquisition, it shows consistency across different acquisitions. Combined with the ubiquity of T₂w imaging scans, the consistency of e-CAMP reconstructions opens the door to massive collections of normalized MR images suitable for machine learning analyses.

Other strategies have been proposed to generate normalized or quantitative images from routine MR images, but e-CAMP is the first to do so with traditional image reconstruction techniques using only physics-based priors. Some previous approaches rely on machine learning strategies, so accuracy depends on an accurate and large training set. S. Qiu, Y. Chen, S. Ma, Z. Fan, F. G. Moser, M. M. Maya, A. G. Christodoulou, Y. Xie, and D. Li, Multiparametric mapping in the brain from conventional contrast-weighted images using deep learning, Magn Reson Med, vol. 87, no. 1, pp. 488-495, January, 2022; L. Feng, D. Ma, and F. Liu, Rapid MR relaxometry using deep learning: An overview of current techniques and emerging trends, NMR Biomed, vol. 35, no. 4, pp. e4416, April, 2022. Their performance with generalization to different equipment or scan parameters is validated case-by-case. Ad hoc normalization strategies similarly must be validated for each new learning goal. J. A. Onofrey, D. I. Casetti-Dinescu, A. D. Lauritzen, S. Sarkar, R. Venkataraman, R. E. Fan, G. A. Sonn, P. 499 C. Sprenkle, L. H. Staib, and X. Papademetris, Generalizable Multi-Site Training And Testing Of Deep Neural Networks Using Image Normalization, Proc IEEE Int Symp 501 Biomed Imaging, vol. 2019, pp. 348-351, April, 2019; R. T. Shinohara, E. M. Sweeney, J. Goldsmith, N. Shiee, F. J. Mateen, P. A. Calabresi, S. Jarso, D. L. Pham, D. S. Reich, and C. M. Crainiceanu, Statistical normalization techniques for magnetic resonance imaging, Neuroimage Clin, vol. 6, pp. 9-19, 2014; R. W. Y. Granzier, N. M. H. Verbakel, A. Ibrahim, J. E. van Timmeren, T. J. A. van Nijnatten, R. T. H. Leijenaar, M. B. I. Lobbes, M. L. Smidt, and H. C. Woodruff, MRI-based radiomics in breast cancer: feature robustness with respect to inter-observer segmentation variability, Scientific Reports, vol. 10, no. 1, pp. 14163, 2020/08/25, 2020; A. Madabhushi, and J. K. Udupa, New methods of MR image intensity standardization via generalized scale, Med Phys, vol. 33, no. 9, pp. 3426-34, September, 2006; L. G. Nyúl, and J. K. Udupa, On standardizing the MR image intensity scale, Magn Reson Med, vol. 42, no. 6, pp. 1072-81, December, 1999; X. Sun, L. Shi, Y. Luo, W. Yang, H. Li, P. Liang, K. Li, V. C. T. Mok, W. C. W. Chu, and D. Wang, Histogram-based normalization technique on human brain magnetic resonance images from different acquisitions, BioMedical Engineering OnLine, vol. 14, no. 1, pp. 73, 2015/07/28, 2015.582; E. Scalco, A. Belfatto, A. Mastropietro, T. Rancati, B. Avuzzi, A. Messina, R. Valdagni, and G. Rizzo, T₂w-MRI signal normalization affects radiomics features reproducibility, Med Phys, vol. 47, no. 4, pp. 1680-1691, April, 2020. By relying on well-established physics, the quantitative mapping method of e-CAMP directly factors out site- and scan-specific details such as echo spacing, flip angle, and B₁ maps at logical points in the reconstruction. Furthermore, there is no need for a training set.

Other normalization methods work by choosing certain image features and making them appear as identical as possible across subjects. If there is any biological variation in these features, that variation is washed out. For example, applying the White Stripe algorithm equalizes white matter values across images, making it difficult to detect the known age-related T₁ and T₂ changes in white matter. R. Bansal, X. Hao, F. Liu, D. Xu, J. Liu, and B. S. Peterson, The effects of changing water content, relaxation times, and tissue contrast on tissue segmentation and measures of cortical anatomy in MR images, Magnetic Resonance Imaging, vol. 31, no. 10, pp. 1709-1730, 2013 Dec. 1, 2013. Strategies to preserve specific biological variables (e.g. age) have been proposed, but these require that all the relevant non-imaging biological variables be known and available from the dataset. J. C. Beer, N. J. Tustison, P. A. Cook, C. Davatzikos, Y. I. Sheline, R. T. Shinohara, and K. A. Linn, Longitudinal ComBat: A method for harmonizing longitudinal multi-scanner imaging data, NeuroImage, vol. 220, pp. 117129, 2020 Oct. 15, 2020. CAMP reconstruction retains all relevant biological variability in the images while removing spurious acquisition effects.

In addition, full validation of the reproducibility of e-CAMP T₂ maps will require many subjects to be repeatedly scanned at different locations with multiple protocols. However, our results do show that e-CAMP T₂ maps from T₂w data achieve good agreement with fully sampled echo train maps and, after Bloch correction, with gold standard maps. Since these have been shown to have high reproducibility across sites, it is expected that e-CAMP T₂ maps will similarly show good agreement across sites. R. M. Gracien, M. Maiworm, N. Brüche, M. Shrestha, U. Nöth, E. Hattingen, M. Wagner, and R. Deichmann, How stable is quantitative MRI?—Assessment of intra-and inter-scanner-model reproducibility using identical acquisition sequences and data analysis programs, Neuroimage, vol. 207, pp. 116364, Feb. 15, 2020; S. C. L. Deoni, S. C. R. Williams, P. Jezzard, J. Suckling, D. G. M. Murphy, and D. K. Jones, Standardized structural magnetic resonance imaging in multicentre studies using quantitative T1 and T2 imaging at 1.5 T, NeuroImage, vol. 40, no. 2, pp. 662-671, 2008 Apr. 1, 2008; N. Weiskopf, J. Suckling, G. Williams, M. Correia, B. Inkster, R. Tait, C. Ooi, E. Bullmore, and A. Lutti, Quantitative multi-parameter mapping of R1, PD*, MT, and R2* at 3T: a multi-center validation, Frontiers in Neuroscience, vol. 7, 2013—Jun. 10, 2013.

It is also likely that the accuracy of e-CAMP maps will vary as a function of acquisition parameters, so a full analysis should test the method on a wider range of acquisition variables. Generally speaking, e-CAMP reconstruction becomes more challenging as (1) the span of TEs becomes narrower, providing less T₂ decay across the data, and (2) the number of k-space lines acquired at each echo becomes smaller, providing less data per image. As these proof of principle studies were acquired at modest resolution, matching these features required some compromise. Compared to higher resolution clinical images, we used a similar number of lines at each echo and a similar span of echo times, albeit with longer echo spacing and shorter echo train length. Experiments with shorter echo spacing could reduce any undesirable diffusion effects, which may be limiting accuracy in the presented studies. H. Y. Carr, and E. M. Purcell, Effects of Diffusion on Free Precession in Nuclear Magnetic Resonance Experiments, Physical Review, vol. 94, no. 3, pp. 630-638, May 1, 1954.

Finally, e-CAMP requires complex-valued, multicoil, k-space data. While most sites do not save these data in the long term, the data can typically be accessed retrospectively within ˜1-7 days of acquisition. In addition, it is feasible to build a pipeline that automatically exports the raw data for processing following the acquisition, storing the resulting T₂ map for future labeling and aggregation. The existing push towards standardized data formats would support this effort across all major vendors.

In conclusion, the algorithms underlying e-CAMP provide the foundation for reconstructing quantitative T₂ maps using only the data acquired in a standard T₂w acquisition. The method can accommodate a range of echo spacings and other protocol variations. Thus, e-CAMP reconstruction may be useful for creating large databases of standardized T₂ maps from heterogeneous and qualitative T₂w image data obtained with site-specific protocols.

e-CAMP with Projected Gradient Descent

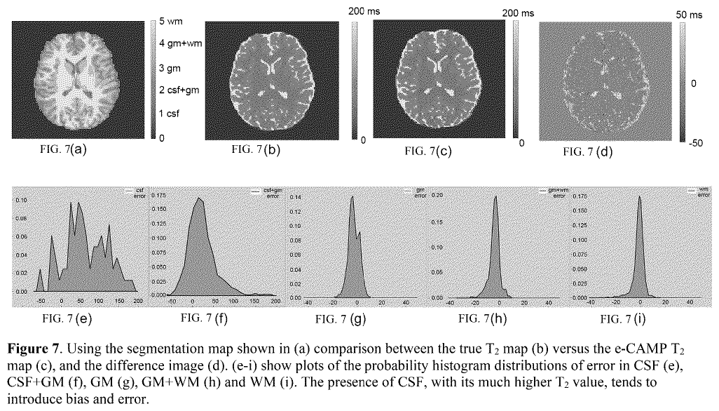

The implementation of e-CAMP described above with reference to FIGS. 1-8, imposes the T₂-decay signal model as a penalty. The multi-echo image series and the T₂ map are alternately optimized with a small penalty coefficient at the first iteration, and then with increasingly larger penalty coefficients at sequential iterations to enforce the model constraint. However, the penalty coefficient and its rate of increase require tedious manual tuning for each reconstruction cycle. Besides, repeated optimization with increasing penalty weights causes slow convergence, up to one hour. In addition, the algorithm is quite sensitive to the accuracy of the phase prior, which was typically estimated from the T₂w reconstruction. A more efficient and robust algorithm is desired to accelerate and simplify the implementation.

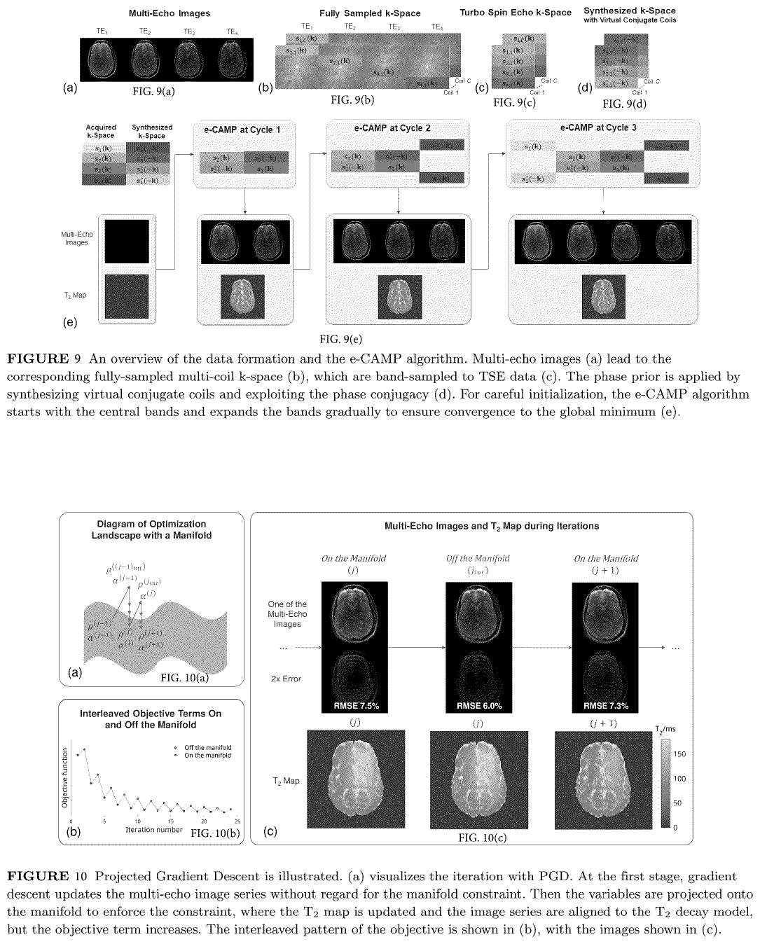

In accordance with an alternate embodiment as disclosed with reference to FIGS. 9-13, the signal evolution model constraint is enforced directly by Projected Gradient Descent (PGD). This embodiment is based on the insight that the feasible solutions, constrained by the signal evolution model, comprise a low-dimensional manifold in the high-dimensional optimization landscape. Projecting the unconstrained intermediate result onto the manifold efficiently handles the constraint without parameter tuning.

Furthermore, this embodiment adopts Virtual Conjugate Coils (VCC) for weaker, but more tolerant, application of the background phase prior. VCC promotes reconstruction of a real-valued image series. Using this approach, violation of the phase prior penalizes the data-consistency term, rather than being completely disallowed.

Sensitive parameter tuning and time-consuming iteration of the penalty method in each k-space band group makes it impractical to generate T₂ maps from T₂w datasets with typical clinical parameters (higher resolution, longer echo train). The algorithmic contributions render efficiency and robustness, and for the first time we show such results.

### VCC

To improve the conditioning of the optimization problem, phase-constrained algorithms are often used to decrease the number of unknowns by half [Willig-Onwuachi Jacob D., Yeh Ernest N., Grant Aaron K., Ohliger Michael A., McKenzie Charles A., Sodickson Daniel K. Phase-constrained parallel MR image reconstruction. Journal of Magnetic Resonance. 2005; 176(2):187-198. 10. Lew Calvin, Pineda Angel R., Clayton David, Spielman Dan, Chan Frandics, Bammer Roland. SENSE phase-constrained magnitude reconstruction with iterative phase refinement. Magnetic Resonance in Medicine. 2007; 58(5):910-921] and generate a real-valued image series \hat{ }ρₚ∈R^{N}. In a T₂w-TSE sequence, the underlying images have almost the same background phase Φ∈C^{N} across echoes as they are refocused by the 180° pulses. The phase of the T₂w image approximately equals Φ and is extracted for subsequent usage. The phase-constrained algorithm is formulated as

$$\\ \begin{matrix}{{min}_{\hat{ρ} , α}}_{} {\sum }_{p = 1}^{P} [ {‖ {s}_{p} - {E}_{p} ( Φ ⊙ {\hat{ρ}}_{p} ) ‖}^{2} + λ TV ( {\hat{ρ}}_{p} ) ] s . t . {\hat{ρ}}_{p + 1} = α {\hat{ρ}}_{p} , & ( 24 )\end{matrix}$$

$$\\ p = 1 , … , P - 1$$

where ⊙ is the Hadamard product.

In practice, however, the phase may not be identical across echoes, e.g. in the presence of imperfect refocusing pulses or stimulated echoes. The estimated phase may also be inaccurate. VCC can be used as a less stringent approach to applying a background phase prior. Blaimer Martin, Gutberlet Marcel, Kellman Peter, Breuer Felix A., Köstler Herbert, Griswold Mark A. Virtual coil concept for improved parallel MRI employing conjugate symmetric signals. Magnetic Resonance in Medicine. 2009; 61(1):93-102; Blaimer Martin, Heim Marius, Neumann Daniel, Jakob Peter M., Kannengiesser Stephan, Breuer Felix A. Comparison of phase-constrained parallel MRI approaches: Analogies and differences. Magnetic Resonance in Medicine. 2016; 75(3):1086-1099. It leverages conjugate k-space symmetry by synthesizing additional virtual coils that have conjugate symmetric k-space data to that of the original coils, as illustrated in FIG. 1(d).

$$\\ \begin{matrix}{s}_{c + C} ( k ) = {s}_{c}^{{}_{} *} ( - k ) , c = 1 , … , C & ( 25 )\end{matrix}$$

where * denotes complex conjugation and k is the k-space index.

Despite the same magnitude as the original coils, the new phase of VCC provides additional encoding information that improves parallel imaging. The background phase is now incorporated in the coil profiles, and only a real-valued image can be perfectly compatible with both the measured and virtual coils. To allow for inaccurate or inconsistent phase Φ, both ρ and α are relaxed to be complex, where ρ contains a slight imaginary component in the VCC framework. The magnitude of α is used to derive T₂(x)=−ΔTE/ln(|α(x)|). Thus, the minimization problem is as follows.

$$\\ \begin{matrix}{{min}_{ρ , α}}_{} J = {\sum }_{p = 1}^{P} [ {‖ ( \begin{matrix}{s}_{p} - {E}_{p} ( Φ ⊙ {ρ}_{p} ) \\ {s}_{p}^{{}_{} *} - {E}_{p}^{{}_{} *} ( {Φ}^{{}_{} *} ⊙ {ρ}_{p} )\end{matrix} ) ‖}^{2} + λ TV ( {ρ}_{p} ) ] s . t . {ρ}_{p + 1} = α {ρ}_{p} , & ( 26 )\end{matrix}$$

$$\\ p = 1 , … , P - 1$$

where E*ₚ=MₚF*C*, and ρ and α are complex.

The band-expanding reconstruction with VCC is illustrated in FIG. 9(e).

### PGD

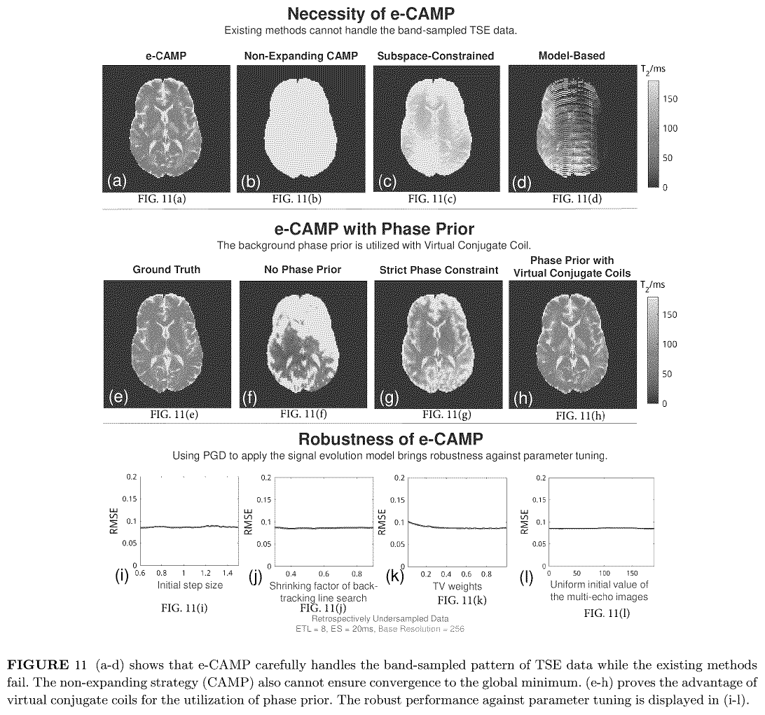

The manifold constrained by the signal evolution model is defined as φ (ρ₁, . . . , ρ_{P}, α), conceptually illustrated in FIG. 10(a). At the first stage of the j-th iteration, ρ^{(j)} is updated to an intermediate result ρ^{(jint)} by gradient descent.

$$\\ \begin{matrix}{ρ}_{p}^{{}_{} ( {j}_{int} )} = {ρ}_{p}^{{}_{} ( j )} - μ {∇}_{{ρ}_{p}} J , p = 1 , … , P & ( 27 )\end{matrix}$$

where μ is determined by backtracking line search.

At the second stage, ρ^{(jint)} and α^{(j)} are projected onto the manifold by minimizing the Euclidean distance. The numerical minimization is with respect to only the first image ρ^{(jint)} 1 and α^{(j)}.

$$\\ \begin{matrix}{{min}_{{ρ}_{1}^{{}_{} ( j + 1 )} , {α}^{{}_{} ( j + 1 )}}}_{} D = {\sum }_{p = 1}^{P} {‖ ( {ρ}_{1}^{{}_{} ( j + 1 )} , {α}^{{}_{} ( j + 1 )} ) - ϕ ( {ρ}_{1}^{{}_{} ( {j}_{int} )} , … , {ρ}_{P}^{{}_{} ( {j}_{int} )} , {α}^{{}_{} ( j )} ) ‖}^{2} & ( 28 )\end{matrix}$$

Specifically, it is implemented by minimizing the disparity between the intermediate results and the signal evolution model while controlling the change of a.

$$\\ \begin{matrix}{{min}_{{ρ}_{1}^{{}_{} ( j + 1 )} , {α}^{{}_{} ( j + 1 )}}}_{} D = {\sum }_{p = 1}^{P} {‖ {ρ}_{p}^{{}_{} ( {j}_{int} )} - {( {α}^{{}_{} ( j )} )}^{p - 1} {ρ}_{1}^{{}_{} ( j + 1 )} ‖}^{2} + {‖ {α}^{{}_{} ( j + 1 )} - {α}^{{}_{} ( j )} ‖}^{2} & ( 29 )\end{matrix}$$

At the third stage, the rest of the image series are aligned to the signal evolution model.

$$\\ \begin{matrix}{ρ}_{p}^{{}_{} ( j + 1 )} = {( {α}^{{}_{} ( j + 1 )} )}^{p - 1} {ρ}_{1}^{{}_{} ( j + 1 )} , p = 2 , … , P & ( 30 )\end{matrix}$$

The general algorithm is summarized in Algorithm 1.

```
Algorithm 1 Projected Gradient Descent e-CAMP with Virtual
Conjugate Coils
  for each band group p = pₛ, . . . , pₑ do
    Initialize the newly incorporated bands and retain the bands
    from the previous group
    Iterate until convergence:
    1. Unconstrained gradient step of J with respect to ρ (Equation
    (27))
    ρ ← ρ− μ∇_{ρJ}
    2. Project onto the manifold by minimizing D (Equations (28)
    and (29)) ρₚ_{ₛ}^{(j+1)}, α^{(j+1)} ← arg min D
    ρₚ_{ₛ}^{(j+1)}, α^{(j+1)}
    3. Align the rest of the image series (Equation (30))
      for p = pₛ + 1 to pₑ do
        ${ρ}_{p}^{( j + 1 )} ⟵ {( {α}^{( j + 1 )} )}^{p - {p}_{s}} {ρ}_{{p}_{s}}^{( j + 1 )}$
      end for
end for
```

### Methods

### Retrospectively Undersampled Human Brain Data

All participants provided informed consent under the approval of the Institutional Review Board (Protocol #2000030538). Fully sampled multi-echo spin-echo human brain data were acquired from one healthy volunteer on a 3 T scanner (MAGNETOM Prisma, Siemens Healthineers, Erlangen, Germany) with a 16-channel head coil. The pulse sequence was performed with TR/TE=2500/20, 40, 60, 80, 100, 120, 140, 160, 180, 200 ms, FOV=220×220 mm², matrix size=128×128, slice thickness=3 mm, slice number=1, bandwidth=219 Hz/pixel, acquisition time=6.5 minutes.

### T₂w-TSE Human Brain Data

TSE human brain data were acquired from one healthy volunteer. The T₂w-TSE data was acquired with the following parameters: ETL=9, ES=20 ms, acquisition time=44 seconds. The other parameters are the same as the retrospectively undersampled dataset. For validation, fully sampled multi-echo data were acquired from the same volunteer with TE=20, 40, 60, 80, 100, 120, 140, 160, 180, 200 ms and other matched parameters. The ground truth T₂ map was derived from the multi-echo fully sampled data using exponential fitting.

Furthermore, a similar pair of T₂w-TSE and multi-echo spin-echo data were acquired with common parameters in clinical practice: matrix size=256×256, ETL 19 and ES=12 ms.

### Coil Processing

The coil sensitivity maps were estimated from the central band(s) of TSE data using ESPIRiT. Uecker Martin, Lai Peng, Murphy Mark J., et al. ESPIRiT—an eigenvalue approach to autocalibrating parallel MRI: where SENSE meets GRAPPA. Magnetic Resonance in Medicine. 2014; 71(3):990-1001. To enable fast reconstruction in the case of large coil numbers, the multi-coil data were compressed [Zhang Tao, Pauly John M., Vasanawala Shreyas S., Lustig Michael. Coil compression for accelerated imaging with Cartesian sampling. Magnetic Resonance in Medicine. 2013; 69(2):571-582.] and the first half of the principal components were retained.

### Reconstruction

Implemented on MATLAB (MathWorks Inc, Natick, MA), the reconstruction was first divided into expanding cycles. In each cycle, the objective function J in equation (26) was minimized with gradient descent as an outer loop. It updated the intermediate results of the image series, where the step size was determined by the Armijo-Goldstein rule. Armijo Larry. Minimization of functions having Lipschitz continuous first partial derivatives. Pacific Journal of Mathematics. 1966; 16(1):1-3. Publisher: Pacific Journal of Mathematics, A Non-profit Corporation. After a step of gradient descent, the intermediate results were projected onto the manifold as an inner loop, as described in Equations (28) and (29). For robust and fast convergence, the projection was implemented with ADAM optimizer. Kingma Diederik P., Ba Jimmy. Adam: A Method for Stochastic Optimization. In: International Conference on Learning Representations (ICLR); 2015; San Diego, CA, USA. The outer loop was bounded by a maximum iteration number, which decreases as the band group expands because the edge bands play a less important role.

With PGD, the remaining few hyperparameters of e-CAMP are the initial step size, the shrinking factor of the back-tracking line search, TV weights, and the initial value of the multi-echo image series. Robustness to the hyperparameters was tested by varying them over a wide range of practical values.

The modern methods for T2 map reconstruction, subspace-constrained and model-based reconstructions [Wang Xiaoqing, Tan Zhengguo, Scholand Nick, Roeloffs Volkert, Uecker Martin. Physics-based reconstruction methods for magnetic resonance imaging. Philosophical Transactions of the Royal Society A: Mathematical, Physical and Engineering Sciences. 2021; 379(2200):20200196. Publisher: Royal Society], were implemented with BART toolbox [Uecker Martin, Ong Frank, Tamir Jonathan I., et al. Berkeley Advanced Reconstruction Toolbox. In: Proceedings of the ISMRM 27th Annual Meeting & Exhibition: 2486; 2015; Toronto, ON, Canada], with the detail in Supplementary Material. A dictionary of 100 T2 values ranging from 10 to 1000 ms was generated for the subspace-constrained method, with l1-norm regularization. There was a total of 100 iterations for reconstruction. The model-based method had 100 inner iterations and 8 Newton steps.

The reconstruction results were evaluated by the normalized root mean square error (NRMSE). Only the parenchyma region was included in the evaluation, excluding the abnormally high T₂ values (T₂>250 ms) from cerebrospinal fluid (CSF).

### Results

FIG. 10(b) shows the interleaved value of the objective function when the variables (ρ and α) are on or off the manifold. The ordinary gradient descent before PGD decreases the objective function value but deviates the variables from the manifold. PGD ensures the variables are on the manifold but at the cost of increasing the objective function value. The overall objective function value is decreased as the iteration proceeds. An example of an intermediate image series and the T₂ maps are shown in FIG. 10(c). The ordinary gradient descent updates the image series (NRMSE from 7.5% to 6.0%), and PGD projects the image series onto the manifold while updating the T₂ map (fewer abnormal pixels) and degrading the image series (NRMSE from 6.0% to 7.3%).

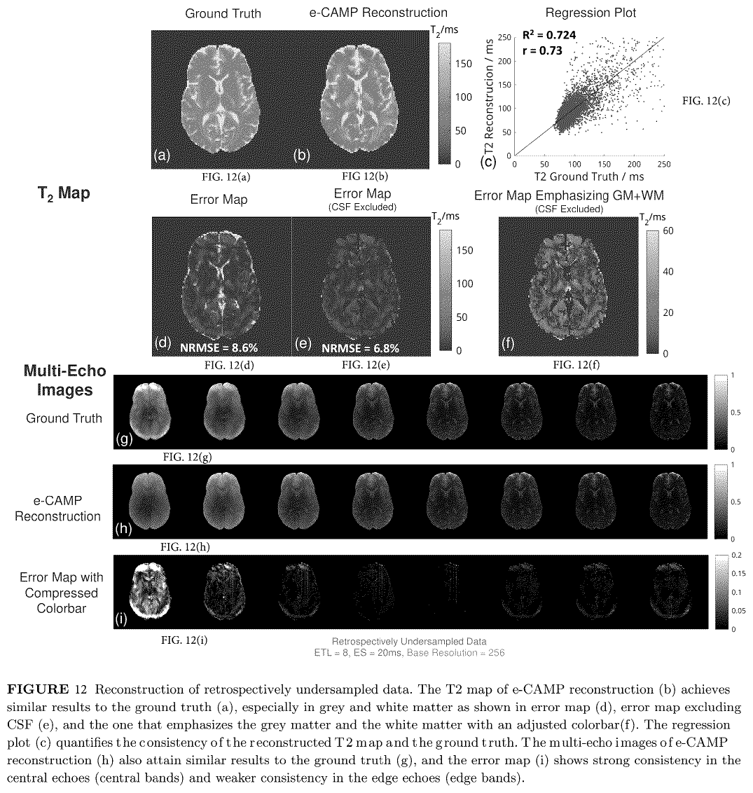

The necessity of e-CAMP using the retrospectively undersampled 8-echo data was demonstrated. While e-CAMP yields a reasonable T₂ map in FIG. 11(a), the non-expanding CAMP in FIG. 11(b) cannot optimize a large number of unknowns simultaneously; neither do the subspace-constrained (FIG. 11(c)) and the model-based (FIG. 11(d)) methods. The advantage of VCC is summarized in FIGS. 11(e-h). The T₂ map is inaccurate without phase prior (FIG. 11(f)) due to poor conditioning. A rigorous real-valued constraint leads to a suboptimal result (FIG. 11(g)). Phase conjugacy with VCC accommodates slight inconsistency of the phase prior and generates a high-fidelity T₂ map (FIG. 11(h)). FIGS. 11(i-l) shows the robustness of e-CAMP to the few remaining hyperparameters as they do not make a significant difference in the reconstruction result, as shown by the nearly constant NRMSE.

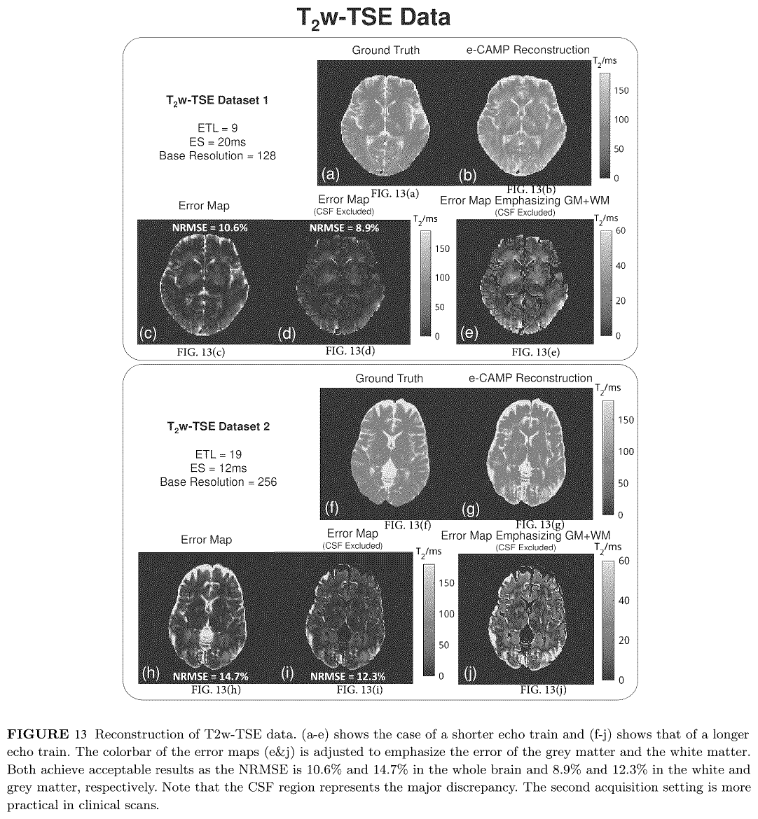

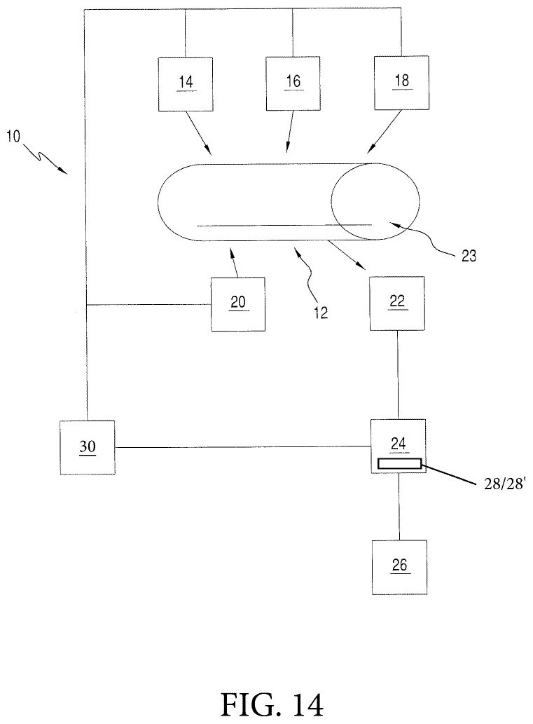

The comprehensive reconstruction results of the retrospectively undersampled 8-echo data are displayed in FIG. 12, which shows a close match to the ground truth especially in white and grey matter, with the coefficient of determination R2=0.728, correlation coefficient r=0.73, whole brain NRMSE=8.6%, white and grey matter NRMSE=6.8%, a common level as reported in many T₂ mapping methods aimed for clinical use. Zhao Bo, Lu Wenmiao, Hitchens T. Kevin, Lam Fan, Ho Chien, Liang Zhi-Pei. Accelerated MR parameter mapping with low-rank and sparsity constraints. Magnetic Resonance in Medicine. 2015; 74(2):489-498; 18. Liu Yuanyuan, Liang Dong, Cui Zhuo-Xu, et al. Accelerating Magnetic Resonance T1 Mapping Using Simultaneously Spatial Patch-Based and Parametric Group-Based Low-Rank Tensors (SMART). IEEE transactions on medical imaging. 2023; 42(8):2247-2261; Liu Bei, Ding Zekang, Zhang Yufei, She Huajun, Du Yiping P. Low-Rank Tensor Subspace Decomposition With Weighted Group Sparsity for the Acceleration of Non-Cartesian Dynamic MRI. IEEE transactions on biomedical engineering. 2023; 70(2):681-693; Heide Oscar, Sbrizzi Alessandro, Berg Cornelis A. T. Cartesian vs radial MR-STAT: An efficiency and robustness study. Magnetic Resonance Imaging. 2023; 99:7-19. While the whole image series (FIG. 4(g-i)) shows high fidelity, the central echo image shows the least structural difference, as it is encoded by the most informative central k-space band. The first echo image shows the most structural difference, because errors in the T₂ map are amplified in the edge echoes, and also because PGD is iterated with regard to the first echo image and the T₂ map, making the first echo image prone to deviation from data fidelity.

Two sets of T₂w-TSE data are shown in FIG. 5. The parameters of the first dataset are less challenging. The reconstructed T₂ map has an NRMSE of 10.6% in the whole brain and 8.9% in the grey and white matter (FIGS. 13(a-e)). The second dataset is more representative of the challenging clinical parameters. e-CAMP still holds its efficacy, with NRMSE=14.7% in the whole brain and 12.3% in the grey and white matter (FIGS. 13(f-j)). The reconstructed T₂ maps are close to the ground truth, though with a slight overestimation of T₂ values at the edge of the brain in the phase encoding direction, due to ringing artifacts from undersampling.

The embodiment disclosed with reference to FIG. 9-13 provides an efficient method to standardize the T₂w images from clinical TSE scans by reconstructing the T₂ maps. The widely used quantitative mapping strategies such as subspace-constrained and model-based methods are accompanied by tailored sampling trajectories, not suitable for the band-sampling pattern of clinical TSE protocols that lead to significantly different intensities across echoes. e-CAMP was explicitly developed for this mission. It promotes data fidelity, piecewise smoothness of images, and adherence to the T₂-decay model. Moreover, the expanding incorporation of k-space bands into the reconstruction scope encourages the escape from spurious local minima. It suggests that the initial bands produce good intermediate results, and the expanding strategy may help other algorithms. However, to Applicant's knowledge, expanding initialization has not been adopted by existing algorithms to show improved performance. For convex objective functions, like that used in subspace-constrained reconstruction, alternate initialization is not expected to change the result.

The previously disclosed embodiment (with reference to FIGS. 1-8) of e-CAMP is based on the penalty method that requires complicated tuning and time-consuming computation. In contrast, e-CMP with PGD (as presented with reference to FIGS. 9-13) has few parameters without the need for careful handling; it also accelerates computation. The algorithm is furthermore enhanced by VCC which promotes real-valued reconstruction results while tolerating the slight deviation of the background phase prior. The produced T₂ maps show high fidelity, and it is the first algorithm to produce T₂ maps with challenging clinical parameters. The implementation takes about 1 minute or less for each of the above-mentioned dataset on a computer with an Intel Core i7-8700 CPU and 32 GB RAM. In general, the complexity of a single iteration scales linearly with the number of slices and the image size. Note that in a long ETL case, the band groups with the edge bands of k-space have fewer iteration steps as they bring in less information, so the processing time is not strictly proportional to ETL, though they are positively correlated.

Like all clinical T₂ mapping, e-CMP with PGD employs spin echo trains to characterize the T₂ decay, and this embodiment focuses on matching the clinical standard (i.e., decay across spin echo trains). Echo train decay differs from gold standard single echo T₂ in that it includes stimulated echoes.

However, the clinical significance of the difference has not been established. One approach to account for this disparity is an EMC framework [Ben-Eliezer Noam, Sodickson Daniel K., Block Kai Tobias. Rapid and accurate T2 mapping from multi-spin-echo data using Bloch-simulation-based reconstruction. Magnetic Resonance in Medicine. 2015; 73(2):809-817], which derives the gold standard T₂ map by matching the multi-echo TSE images to a Bloch-simulated dictionary created using experimental parameters such as flip angle and T₁. The prior embodiment of e-CAMP shows that matching the e-CAMP reconstructed image series to an EMC dictionary, even using nominal parameters, matches gold standard T₂ values. The T₂ map of the brain may be contaminated by ringing artifacts due to the undersampling. This artifact is expected to be more obvious in certain organs, such as abdominal or prostate imaging, where ringing from fat can be quite pronounced. Advanced regularization terms may be necessary to suppress these ringing artifacts.

e-CAMP with PGD provides for efficient standardization of clinical T₂-weighted images to a method that requires neither parameter tuning nor a highly accurate phase prior. The proposed algorithm enforces the T₂-decay model by Projected Gradient Descent and implements the phase prior by Virtual Conjugate Coils. It is robust to hyperparameters, and the reconstruction results are close to the ground truth, even for a long echo train and short echo spacing. The proposed algorithm is promising to utilize the massive TSE data in clinical scanners.

Both e-CAMP and e-CAMP with PGD are implemented in traditional MRI systems. As shown with reference to schematic of Figure ##, a superconducting magnet 14 of the MRI system 10 applies a spatially uniform and temporally constant main B₀ magnetic field. Further, excitation of nuclear spin magnetization within the examination volume of the magnetic housing 12 is applied by RF coils 20. More particular, the RF coils 20 apply a radio frequency pulse sequence, the B₁ field, which is superimposed perpendicular to the B₀ field at an appropriate proton resonant frequency.

These components of the MRI system 10 are combined with a plurality of magnetic gradient coils 18 that apply nonlinear magnetic gradient fields to the examination volume of the magnetic housing 12. The nonlinear magnetic gradient fields facilitate spatial encoding of the nuclear spin magnetization. During MRI procedures, pulse sequences composed of magnetic gradient fields (applied by the magnetic gradient coils 18) and radio frequency fields (applied by the RF coils) are applied to a targeted subject (such as a live patient) while subject to the temporally constant main B₀ magnetic field (applied by the superconducting magnetic 14) to generate magnetic resonance signals, which are detected, stored and processed to reconstruct spectra and images of the object. These procedures determine the characteristics of the reconstructed spectra and images such as location and orientation in the targeted subject, dimensions, resolution, signal-to-noise ratio, and contrast. The operator of the magnetic resonance device typically selects the appropriate sequence, and adjusts and optimizes its parameters for the particular application.

As briefly explained above, the MRI system 10 includes a traditional magnet housing 12, a superconducting magnet 14 generating the magnet field B₀ to which the patient is subjected, shim coils 16, RF coils 20, receiver coils 22, and a patient table 23. The MRI system 10 also includes gradient coils 18 creating nonlinear magnetic gradient fields.

As is well known in the art, the superconducting magnet 14 produces a substantially uniform magnetic B₀ field within its design field of view (FOV). This B₀ field is directed along the positive Z-axis. As for the gradient coils 18, the present MRI system 10 employs a plurality of such gradient coils.

The MRI system 10 also includes measurement circuitry 24 producing data used to reconstruct images displayed on a display 26. The algorithm of either e-CAMP 28 or e-CAMP with PGD 28′ is integrated with the measurement circuitry 24 to produce images in accordance with the present invention. Preferably, the application of control signals is achieved via a control system 30 linked to the various operational components of the MRI system 10 under the control of an operator of the MRI system 10.

While the preferred embodiments have been shown and described, it will be understood that there is no intent to limit the invention by such disclosure, but rather, is intended to cover all modifications and alternate constructions falling within the spirit and scope of the invention.

1. An imaging method, comprising:
calculating T₂ maps using only data from a T₂w image scan.

2. The method according claim 1, wherein calculating uses an exact but linear expression to capture an exponential decay model.

3. The method according claim 1, wherein calculating is biconvex so that it can be alternately minimized.

4. The method according claim 1, wherein calculating applies a stepwise initialization process that avoids spurious local minima due to the significant gaps in k-space.

5. The method according claim 1, wherein calculating includes a signal evolution model constraint that is enforced directly by projected gradient descent.

6. The method according claim 1, wherein calculating includes virtual conjugate coils.

7. An MRI system, comprising:
a magnet housing;
a superconducting magnet;
shim coils;
RF coils;
receiver coils;
a patient support;
and measurement circuitry producing data used to reconstruct images displayed on a display, wherein the measurement circuitry integrates either expanding-constrained alternating minimization for parameter mapping or expanding-constrained alternating minimization for parameter mapping with projected gradient descent.

8. The MRI system according to claim 7, wherein expanding-constrained alternating minimization for parameter mapping or expanding-constrained alternating minimization for parameter mapping with projected gradient descent calculates T₂ maps using only data from the T₂w image scan.

9. The MRI system according to claim 7, wherein expanding-constrained alternating minimization for parameter mapping or expanding-constrained alternating minimization for parameter mapping with projected gradient descent uses an exact but linear expression to capture an exponential decay model.

10. The MRI system according to claim 7, wherein expanding-constrained alternating minimization for parameter mapping or expanding-constrained alternating minimization for parameter mapping with projected gradient descent is biconvex so that it can be alternately minimized.

11. The MRI system according to claim 7, wherein expanding-constrained alternating minimization for parameter mapping or expanding-constrained alternating minimization for parameter mapping with projected gradient descent applies a stepwise initialization process that avoids spurious local minima due to the significant gaps in k-space.

12. The MRI system according to claim 7, wherein expanding-constrained alternating minimization for parameter mapping with projected gradient descent includes a signal evolution model constraint that is enforced directly by projected gradient descent.

13. The MRI system according to claim 7, wherein expanding-constrained alternating minimization for parameter mapping with projected gradient descent includes virtual conjugate coils.
